(19) United States
(12) Patent Application Publication
(10) Pub. No.: US 2016/0015916 A1
(43) Pub. Date: Jan. 21, 2016

(54) POSITIVE AIRWAY PRESSURE SYSTEMS

(71) Applicant: HANCOCK MEDICAL, INC., Mountain View, CA (US)

(72) Inventors: Thomas G. GOFF, Mountain View, CA (US); Nathaniel L. BOWDITCH, Menlo Park, CA (US)

(21) Appl. No.: 14/773,236

(22) PCT Filed: Mar. 12, 2014
(86) PCT No.: PCT/US2014/024113

§ 371 Date: Sep. 4, 2015

Related U.S. Application Data
(60) Provisional application No. 61/794,736, filed on Mar. 15, 2013.

(51) Int. Cl. A61M 16/00; A61M 16/10; A61M 16/06

(52) U.S. Cl. CPC A61M 16/0057; A61M 16/06; A61M 16/105; A61M 2205/02; A61M 2205/75

(57) ABSTRACT

Described here are positive airway pressure (PAP) systems and methods of using the same. The systems may comprise an air delivery matrix configured to deliver air from a chassis or housing and a user interface such as a mask. The systems may be wearable on a head of a patient, and in some instances may include an air bladder positioned between the chassis or housing and the head of the patient.

### FIELD

The invention is generally directed to Positive Airway Pressure (PAP) devices and methods of using and controlling these devices.

### BACKGROUND

During sleep, all muscles, including those of the upper airway, lose tone and relax. Obstructive Sleep Apnea (OSA) occurs when tissue blocks the upper airway during sleep. This will cause a drop in blood oxygen and a rise in blood carbon dioxide. The brain will sense these changes, and awaken the person enough to restore muscle tone to the structures of the upper airway, and the airway will reopen.

The severity of OSA is determined by the number of blockages per hour of sleep, also called the apnea-hypopnea index (AHI). These include complete blockages (apneas) and partial blockages (hypopneas). The severity of OSA, as determined by a sleep study, is classified as follows:

| SEVERITY | BLOCKAGES PER HOUR |
|---|---|
| Mild | 5-15 |
| Moderate | 15-30 |
| Severe | 30+ |

OSA disrupts restorative sleep. Chronic fatigue has long been recognized as the hallmark of OSA. But more recently, large clinical studies have shown a strong link between OSA and stroke and death. This link is independent of other risk factors for cardiovascular disease such as hypertension, obesity, high cholesterol, smoking and diabetes.

As discussed above, several structures can cause blockage of the upper airway: the tongue, the soft palate, the uvula, the lateral walls of the pharynx, the tonsils and the epiglottis. In most patients, the blockage is caused by a combination of these anatomical structures.

Many current procedures and devices have been used to stabilize, modify or remove tissue in the airway to treat OSA. In uvulopalatopharygoplasty (UPPP), the uvula, part of the soft palate and the tonsils are removed. A Repose stitch is used to tie the tongue to the mandible to prevent its posterior movement. Oral appliances move the mandible forward (very slightly) to create more space in the airway.

None of these approaches has achieved much more than a 50% success rate, with success defined as a 50% decrease in AHI to a score below 20. The limited success of these approaches likely stems from the fact that they don't address all anatomical sources of a blockage.

The most widely used therapeutic system for OSA is a PAP system such as a continuous positive airway pressure (CPAP) system. A CPAP system usually consists of three parts: a user interface forming a largely airtight seal over the nose or nose and mouth, an air pressurizing housing or console and an elongated tube connecting the two. The user interface contains one or more holes, usually at the junction with the tube. A CPAP system works by pressurizing the upper airway throughout the breathing cycle, essentially inflating the airway to keep it open. A CPAP system thus maintains a pneumatic splint throughout the respiratory cycle.

Unlike interventions that treat specific blockages, a CPAP system addresses all potential blockage sites. The success rate in patients (dropping AHI by >50%) exceeds 80%, and its cure rate (decreasing AHI below 5) is close to 50%. The drawback to a CPAP system is poor patient compliance, i.e. continuous use by the patient. In one large study, only 46% of patients were compliant with a CPAP system, even though compliance was defined as using the CPAP system at least 4 hours per night at least 5 nights per week.

Critical pressure is the airway pressure a given patient requires to maintain an open airway during sleep. Critical pressure is measured in cm of water, and will typically be between 6 and 14 cm of water for a patient requiring CPAP. In a given patient, the efficacy of a CPAP system goes up as pressure is increased. But, as higher pressure makes the CPAP system more uncomfortable to the patient, patient compliance drops. The goal of the healthcare professional in setting up a CPAP system for a patient is to achieve critical pressure without exceeding it. This will make the CPAP system both effective and tolerable.

In a given patient, there are several factors that affect critical pressure. The pressure supplied by the CPAP system necessary to achieve critical pressure varies through the breathing cycle. When a patient is exhaling, the patient is supplying some air pressure to the airway, and thus requires limited pressure from the CPAP system to maintain critical pressure. But when the patient is inhaling, he is decreasing pressure in the airway. During inhalation, more pressure is required by the CPAP system to maintain critical pressure in the airway. There are now many available CPAP systems that monitor the respiratory cycle, and provide less pressure during the portions of the respiratory cycle when less external pressure is required to maintain critical pressure in the airway. Such adaptive systems, which include systems commercially known as BiPAP and C-Flex, make CPAP systems more comfortable, improving the compliance of many patients. So, during the respiratory cycle, critical pressure does not change. But the pressure contributed by the CPAP system to maintain critical pressure changes during the respiratory cycle.

Critical pressure can change based on sleeping position in many patients. Critical pressure will usually be higher when a patient is in a supine position (i.e. on his back) than when a patient is in a lateral position (on his side). This is because many of the structures that can block the airway, such as the tongue and uvula, are anterior to the airway. When a patient is in a supine position, gravity pulls these structures toward the airway, and a greater pressure (critical pressure) is required to keep the airway open. When a patient is in a lateral position, gravity is not pulling these structures directly into the airway, and thus less pressure is required to maintain an open airway. This was demonstrated in a study published in 2001 (Penzel T. et al. 2001. Effect of Sleep Position and Sleep Stage on the Collapsibility of the Upper Airways in Patients with Sleep Apnea; SLEEP 24(1): 90-95.). Additionally, most sleep studies used to diagnose OSA will track body position and will determine whether a patient has airway blockages more frequently when sleeping in a supine position. Other sleep studies have found that the lateral position results in fewer observed apneas than the supine position. (Cartwright R. et al. 1984 Effect of Sleep Position on Sleep Apnea Severity: SLEEP 7:110-114). (Pevernagie D. et al. 1992 Relations Between Sleep Stage, Posture, and Effective Nasal CPAP Levels in OSA: SLEEP 15: 162-167). Further studies have shown that apnea events in the supine position tend to be more severe, have longer duration, be accompanied by a greater oxygen desaturation and increased heart rate, and be more likely to result in arousals and awakenings. (Oksenberg A. et al. 2000 Association of Body Position with Severity of Apneic Events in Patients with Severe Non-positional Obstructive Sleep Apnea: CHEST 118: 1018-1024).

A common complaint with PAP systems is their noise level. This noise may include several sources—notably the motor of the airflow generator and the noise created by the air moving through the system. Current attempts to limit the noise of airflow generators include the use of highly efficient, well engineering bearings with low resistance, to low inertia impellers to reduce the motor load when speed and pressure changes are desired. Although these technologies may help reduce the noise caused by the airflow generators, they do not help reduce the noise caused by the movement of air through the system. This airflow noise can, in many cases, be louder and more disruptive than the noise from the airflow generator. In this realm, classic CPAP tubing has significant shortcomings. Its plastic walls do little to absorb the noise created by the airflow. Additionally, turbulence in the airflow is a major contributor to this noise. Transitions, joints, junctions and changes in cross sections all result in added turbulence, which contributes added noise.

### SUMMARY

Described here are positive airway pressure (PAP) systems and methods for treating a patient with OSA or other breathing problems. The positive airway pressure systems may comprise a system housing or chassis, a user interface (such as a mask or the like), and an air delivery matrix configured to deliver air to the user interface, which in turn may deliver positive airway pressure to the user. The housing may comprise a pressure source configured to deliver air to the user interface via the air delivery matrix.

In some variations, the positive airway pressure systems may be configured to be mounted or otherwise secured to a patient's head. Specifically, the housing or chassis may be configured to be secured to a patient's head. In some of these variations, the system may comprise an air bladder configured to be positioned between the housing and the patient's head. In some instances when the system is configured to be mounted or otherwise secured to a patient's head, the system may include a sensor that is mounted onto the top of the patient's head. In some of these variations, an output pressure of a pressure source may be controlled using a signal from the sensor.

As mentioned above, the PAP systems described here may include at least one housing. The housing may have an interior configured to receive one or more system components and may be configured to be secured to the top of the patient's head, preferably in a median position. One system component may be a gas pressure source, such as a compressor, which may have a controllable output pressure and may be disposed within the interior of the housing and preferably has a controllable motor drive. The gas pressure source has an outlet opening which is configured to be connected to an air delivery matrix that preferably leads to a user interface which is configured to sealingly fit over the patient's nose and/or mouth. The PAP system has a harness assembly or system chassis which is configured to secure at least part of the system to the top of the patient's head.

As mentioned above, in some variations the PAP systems described have may have a sensor, such as an accelerometer, that may be secured within or to a system chassis or on or to the harness assembly that is configured to secure at least part of the PAP system to the user's head. In some embodiments a head position sensor is configured to sense the position of the patient's head with respect to a reference plane (e.g. a horizontal plane) and to generate a signal representing the sensed position of the patient's head. A suitable accelerometer is a Freescale 3-Axis MEMS Accelerometer (MMA845XQ), particularly the MMA8453Q model, which generates a signal series representing a three axis orientation with respect to gravity.

The systems described here may also comprise a controller, preferably a microprocessor such as Atmel AVR Model # ATMMega328, which may be provided to control the PAP system so as to adjust the output of the gas pressure source according to the sensed position of the patient's head to provide a critical pressure to the patient's airway passage to maintain patency. The controller is preferably secured within the system's chassis and is configured to receive the head position signals transmitted from the head position sensor. In instances where the system comprises a head position sensor, the controller may be configured to receive the head position signals and to determine a suitable pressure source output pressure for the particular sensed head position from the received head position signal. The controller preferably has a stored relationship between sensed head position and suitable pressure source output pressure and is configured to generate a control signal for the pressure source representing the determined suitable gas pressure. The control signal generated by the controller is transmitted to the pressure source, e.g. the driving motor of a compressor, to control the output of the pressure source so as to provide the determined suitable gas pressure for the sensed position of the patient's head.

In one embodiment, a pressure sensor senses the actual gas pressure directed to or received by the user and the controller compares the directed or received sensed pressure with the desired or determined suitable pressure and adjusts the control signal to the pressure source so as to provide the pressure source output that provides the critical pressure that maintains patency in the patient's airway passage.

Alternatively, the pressure source may be operated at a constant pressure level and a control valve disposed between the pressure source and the patient's user interface receives the control signal to control the output pressure. The control valve may be provided in the compressor outlet, in a gas flow line to the patient's user interface or in the patient's user interface, to provide the determined suitable gas pressure to the patient that maintains patency in the patient's airway passage.

Although the accelerometer and the controller are described herein as two separate devices, they may be combined into a single device.

The controller may be configured to determine suitable compressor output pressures from the head position signal-pressure source output pressure relationship for at least two patient head positions, one head position may be a supine position and another second patient position might be a lateral position, preferably at least 20° from the supine position. In one embodiment, the controller may be provided with a readable library listing a plurality of head position signals with corresponding suitable pressure source output pressures. In another embodiment, the controller has a preprogrammed algorithm representing a relationship between head position signal and corresponding suitable pressure source output pressure. The microprocessor is configured to use the received head position signals to calculate from the algorithm suitable pressure source output pressures and generate suitable control signals for the pressure source. The relationship between the head position signal and suitable pressure source output pressure may be a stepped function, e.g. two or more positions with suitable pressure source output pressure or a continuous function. There may be a gradual change in pressure between stepped functions. The continuous function preferably has a maximum rate of change in the pressure source output pressure with respect to head position with head positions between about 30° and 60° from the supine position (0°).

The set point for a suitable pressure source output pressure for one of the head positions, e.g. the supine position, may be set by a health professional based upon the patient's sleep study. The set point for other position may also be set by the health professional.

In one embodiment, the supine position may be defined as within 30° of vertical, with vertical being the sleeping position where the nose is pointed directly upward, orthogonal to the sleeping plane which is horizontal.

The pressure source is preferably a rotary blower such as the Nidec Copal TF037C, which has a turbine and a controllable drive motor. An alternative pressure source may be a bellows system which maintains a pressurized storage tank that provides pressurized breathing gas to the patient's user interface. Other gas pressure sources may be utilized.

The electrical power source for the pressure source drive motor is preferably a portable power source component such as one or more batteries which may be provided within the system housing. However, the electrical power source may be an electrical power cord for connection to an electrical source (e.g. a wall outlet or a separate battery pack) and may be provided to directly supply electrical power to the pressure source and/or to recharge one or more batteries. The electrical power source is connected to and powers the drive motor that controls the output pressure of the pressure source such as a rotary compressor.

In one embodiment, the system housing may be one or more separate housings or system chasses, and is mounted on top of a patient's head or on the forehead, preferably in a medial position, by one or more straps. Other means to secure the system housing or system chassis to the patient's head may be used.

Initially, a health professional may set the output pressure of the compressor for one or more of the patient's head positions that have been based upon a sleep study performed on the patient. The second head position should be at least 20° away from the first head position. Optionally, the health professional may also set the set point for the output pressure of the system when the patient's head is at other positions. Preferably, the controller is programmed to select a suitable compressor output pressure from a preset table or library for at least one head position or determine a suitable pressure from a preprogrammed algorithm that is based upon the sensed position of the patient's head. The algorithm defines the relationship between the head position signal and the suitable compressor output pressure.

With the PAP system mounted on the patient's head, the head position sensor may first be calibrated, preferably when the patient's head is in a supine position. The calibrated head position sensor senses the patient's head position and generates a sensed head position signal which is transmitted to the controller. The controller determines a suitable pressure source output pressure for the sensed head position signal and generates a control signal for the pressure source to provide the suitable pressure output pressure. In one embodiment, the controller compares the determined suitable pressure source output pressure with the current pressure output of the pressure source and if they differ by a specified amount, the controller generates a new control signal for the pressure source. If they do not differ by the specified amount the system loops back and continues to monitor the patient's head position.

The pressure source output pressure requirements can vary between a low point at exhalation to a high point at inhalation for each position of the patient's head which forms an output pressure envelope for the patient.

During sleep, the position of the patient's head can be the most important determinant of critical pressure for the patient since the anatomical structures that might block the airway (such as the tongue, the soft palate, the uvula and the tonsils) are in the head. Thus, the position sensor that determines the position of the head can be valuable in effectively controlling the pressure output of a PAP system.

In one embodiment the PAP system provides a first (higher) gas pressure from the compressor when the patient's head is in a supine position and a second (lower) pressure when the patient's head is in a lateral position. The gas pressure supplied to the patient when the patient's head is in a lateral position would likely be 1-8 cm of water less than the pressure supplied when the patient's head is in the supine position. Additionally, the PAP system can vary pressure more continuously based on several patient sleeping positions. With such a system, higher pressure would be supplied to the patient by the pressure source the closer a person's head is to a completely supine position with the patient's nose lying in a vertical plane. Slightly lower pressure could be supplied, for example, if a person's head was 20° off from the supine position and other positions further away from the supine position. The lowest gas pressure would usually be when the patient's head is in a lateral position 90° or more from the supine position.

The patient's head positions are described herein primarily in terms of the supine position, a lateral position and positions between these two positions about a longitudinal axis passing through the patient's head. The head position sensor may also sense when the patient's head is tilted toward or away from the patient's chest, or rotated further than a lateral position 90° away from the supine position. A patient whose head is tilted far forward during sleep (i.e. the chin is close to the chest), may experience an even higher frequency of airway blockages than when in a supine position and may need a higher gas pressure to maintain an open airway passage than when in a supine position.

The PAP system which modulates its output pressure based on a patient's head position while sleeping could also be used to determine whether a given patient's sleep apnea event frequency and severity are affected by sleeping position, and PAP system output could be modulated accordingly. For example, the PAP system pressure may be lowered from about 11 cm (of water) to about 9 cm as the patient moved from a supine to a lateral sleeping position. The system could also further modulate pressure output based upon whether the number of airway blockages increased or decreased (e.g. as measured by pressure sensors in the PAP user interface or within the gaseous flow from the compressor to the PAP user interface) and the corresponding patient position.

The controller of the PAP system may be used to provide different pressure source output pressures within the pressure envelope at different points in the respiratory cycle at any given patient head positions. For example, higher pressure source output pressures may be provided during inhalation and lower pressure source output pressures during exhalation to maintain the critical pressure within the patient's airway passage.

In some variations of the PAP systems described here, the PAP system may provide different suitable gas pressures depending on the head position of the patient. This would improve patient comfort while providing the critical pressure at different positions to maintain an open airway. Additionally, since patients prefer lower PAP pressures, such a system might also cause patients to prefer to sleep in positions (such as a lateral position) that cause the system to provide a lower gas pressure to the patient. The lower output pressure would also spur the patient to sleep in a position that leads to fewer airway blockages. The lower output pressure would tend to disturb a person's sleep much less because of comfort of lower pressure and less noise due to the slower operation of the compressor drive motor.

In some variations where the PAP systems described here are wearable, the wearable PAP system may preferably include one or more housings which insulate the patient's head from heat, sound and vibration from the from the PAP system. For example a pad may be positioned at the bottom of the housing(s) or the bottom of the housing may be spaced from the patient's head to minimize such heat, noise and vibrations. The drive motor for the compressor may have additional vibration and noise dampers to produce a less disturbing operation. Materials such as foams, gels, plastic members, rubber-like members or contained fluids may be used to isolate and/or reduce the noise and vibrations which emanate from the pressure source.

Further, in some variations a wearable PAP system may be spaced from the top of the patient head and may be supported by pod extensions or feet which are secured to the harness assembly. The bottoms of the pod extensions or feet are preferably padded to minimize patient discomfort. Because a substantial portion of the bottom surface of the housing is spaced away from the patient's head, trapped heat underneath the bottom of the housing may be minimized. The open space between the patient's head and the bottom of the housing(s), in addition to providing ventilation also reduces noise and vibration from the system housing to the patient.

Moreover, because a wearable PAP system as described here may not have a long tube connecting the patient's user interface to a remote pressure generating unit, there may be fewer restrictions on a patient's movement during sleep. There may also be a reduced likelihood of pulling the user interface away from its operative position on the patient's face, and there is no remote pressure generating unit that might be pulled off an adjacent night stand. Examples of the systems and methods described here will be discussed in more detail below.

### BRIEF DESCRIPTION OF DRAWINGS

FIG. 1 shows a perspective view of a variation of the PAP systems described here.

FIG. 2 is a perspective view of a PAP system shown in FIG. 1 with a user interface mounted over the patient's nose and the housing mounted on the patient's head.

FIG. 3 is a rear view of the PAP system shown in FIG. 2.

FIG. 4 is a side elevational view of the system shown in FIG. 1 with portions of the system housing removed to illustrate the various components within the system housing.

FIG. 5 is a top view of the system with portions of the system housing removed to illustrate the various components within the system housing.

FIG. 6 is a block diagram illustrating the system shown in FIG. 1.

FIG. 7 is a flow diagram illustrating position calibration of the system processing.

FIG. 8 is a flow diagram illustrating processing for a variation of the PAP systems described here.

FIG. 9 is a flow diagram illustrating system processing using scalar measurement.

FIG. 10 is a top view of the PAP system shown in FIG. 2 with the patient's head in the supine position and the patient's nose pointed directly up, orthogonal to the sleeping plane.

FIG. 11 is a top view of the PAP system with the patient's head less than 20° from the supine position.

FIG. 12 is a top view of the PAP system with the patient's head about 30°-60° from the supine position.

FIG. 13 is a top view of the PAP system with the patient's head in the lateral position 90° from the supine position.

FIG. 14 is a top view of the PAP system with the patient's torso in a lateral position and the patient's head in a position greater than 60° and less than 90° from the supine position.

FIG. 15 is a top view of the PAP system with the patient's torso in a lateral position and the patient's head in a position greater than 90° from the supine position.

FIG. 16 shows a side view of a user with the head tilted down, bringing the chin closer to the user's chest.

FIG. 17 shows a side view of a user with the head tilted up, moving the chin further away from the user's chest.

FIG. 18 illustrates how different algorithms can relate head position input signals (i.e. representing degrees from a supine position) and suitable pressure source output pressures.

FIG. 19 is a graph schematically illustrating output pressure variations within a pressure envelope for a supine and a lateral head positions.

FIG. 20 is an exploded perspective view of a variation of a housing for a PAP system showing how the components are fit into the interior of the housing.

FIG. 21 illustrates how the housing shown in FIG. 20 may interface with a replaceable strap structure or harness assembly for securing the PAP system to the head of a patient or user. The shaded contact interface securely attaches the PAP system to the strap structure.

FIG. 22 is a perspective view which depicts a variation of a PAP system which has multiple housings to contain the various components of the system.

FIG. 23 is a perspective view showing a variation of a PAP system with multiple housings that are interconnected by a fabric or mesh that is integral with the harness assembly.

FIG. 24 is a perspective view that illustrates a variation of a PAP system having multiple housings interconnected by hinged, semi-rigid elements.

FIG. 25 illustrates a variation of the wearable CPAP systems described here, including a chassis system, air delivery matrix, and user interface.

FIG. 26A depicts a perspective view of a variation of an air delivery matrix as described here. FIG. 26B shows a cross-sectional view of the air delivery matrix of FIG. 26A.

FIG. 27A shows another embodiment of an air delivery matrix as described here, tapering narrower from top to bottom.

FIG. 27B shows another embodiment of an air delivery matrix as described here, tapering narrower from bottom to top.

FIG. 28A shows an embodiment of an air delivery matrix as described here, which may be largely uniform throughout its length.

FIG. 28B shows an embodiment of an air delivery matrix as described here, which has two portions at the top which merge into one portion at the bottom.

FIG. 28C shows an embodiment of an air delivery matrix as described here, which has one portion at the top which separates into two portions at the bottom.

FIG. 28D shows an embodiment of the air delivery matrix which has two portions at the top which merge in one portion at the center and then separate into two portions again at the bottom.

FIG. 29A-29E illustrates cross-sections of variations of the air delivery matrixes described here having different geometries.

FIG. 29F illustrates a cross-section of a variation of an air delivery matrix as described here with two sections of inner material contained by the outer material.

FIG. 29G illustrates a cross-section of a variation of an air delivery matrix as described here with three sections of inner material contained by the outer material.

FIG. 29H illustrates a cross-section of a variation of an air delivery matrix as described here including two lumens for pressure measurement or placement structural support elements.

FIG. 29I illustrates a cross-section of a variation of an air delivery matrix as described here including one lumen for pressure measurement or placement structural support elements.

FIG. 29J illustrates a cross-section of a variation of an air delivery matrix as described here including three lumens for pressure measurement or placement structural support elements.

FIG. 30A illustrates a cross-section of a variation of an air delivery matrix as described here including one lumen configured for pressure measurement, one lumen configured for placement of structural support elements, and one lumen configured for either pressure measurement or placement of structural support elements.

FIG. 30B illustrates a cross-section of a variation of an air delivery matrix as described here including one lumen configured for pressure measurement, and two lumens configured for placement of structural support elements.

FIG. 30C illustrates a variation of an air delivery matrix as described here including one lumen configured for placement of structural support elements, and two lumens configured for either pressure measurement or placement of structural support elements.

FIG. 30D illustrates a variation of an air delivery matrix as described here including a central lumen configured for placement of structural support elements and a lumen configured for pressure measurement.

FIG. 30E illustrates a variation of an air delivery matrix as described here including a first central lumen configured for placement of structural support elements and a second central lumen configured for pressure measurement.

FIG. 31 is a perspective view of a variation of a portion of the PAP systems described here including an air delivery matrix and a user interface in which the air delivery matrix comprises lumens of varying lengths.

FIG. 32 is a perspective view of a portion of a variation of a system chassis suitable for use with the PAP systems described here.

FIG. 33 is a cross-section of a variation of the portion of the system chassis through A-A of FIG. 32 showing a portion of the internal components.

FIG. 34 is a cross-section of a variation of the portion of the system chassis through B-B of FIG. 32 showing a portion of the internal components.

FIG. 35 is a cross-section of a variation of the portion of the system chassis through A-A of FIG. 32 showing a portion of the internal components.

FIG. 36 is a cross-section of a variation of the portion of the system chassis through A-A of FIG. 32 showing a portion of the internal components.

FIG. 37 is a cross-section of a variation of a portion of the system chassis through A-A of FIG. 32 showing a portion of the internal components.

FIG. 38 is a cross-section of a variation of a portion of the system chassis through B-B of FIG. 32 showing a portion of the internal component.

FIG. 39 is a perspective view of a variation of a system chassis suitable for use with the PAP systems described here, showing the air inlets in one embodiment.

FIGS. 40A-40C depict perspective views of variations of the air bladders described here.

FIGS. 41A-4C depict top views of variations of the air bladders described here.

FIG. 42 is a top view of a variation of the air bladders described here showing a manual inflation/deflation mechanism.

FIG. 43 is a side cross-sectional view of a portion of a variation of a system chassis as described here illustrating an air bladder inflated by a blower.

FIGS. 44 and 45 depict perspective view of variations of the system chasses described here.

### DETAILED DESCRIPTION

Described here are positive airway pressure (PAP) devices, systems, and methods. The devices, systems, and methods described may include wearable PAP systems and may be configured in a manner to increase the ease of use, efficacy, comfort, ease of manufacture, safety, and/or durability of the system. It should be appreciated that aspects of the systems, devices, and methods described here may be incorporated into or otherwise used with a CPAP system having a bedside console. While a number of embodiments will be described here by way of illustration and example, certain changes and modifications may be practiced without departing from the scope of the devices, systems, and methods described here.

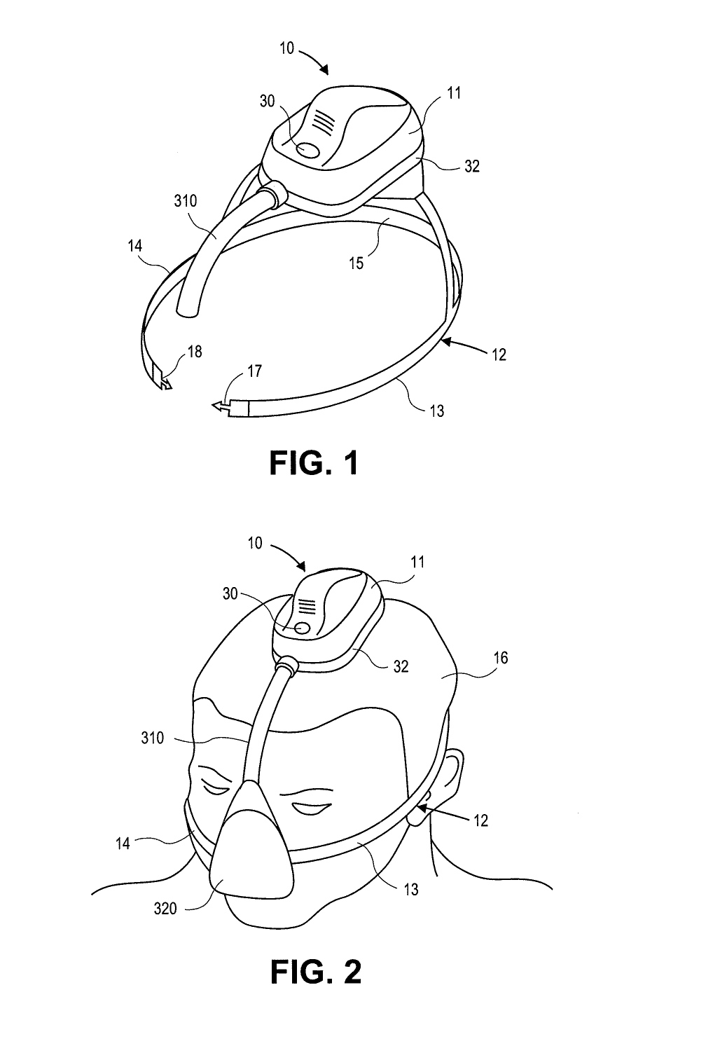

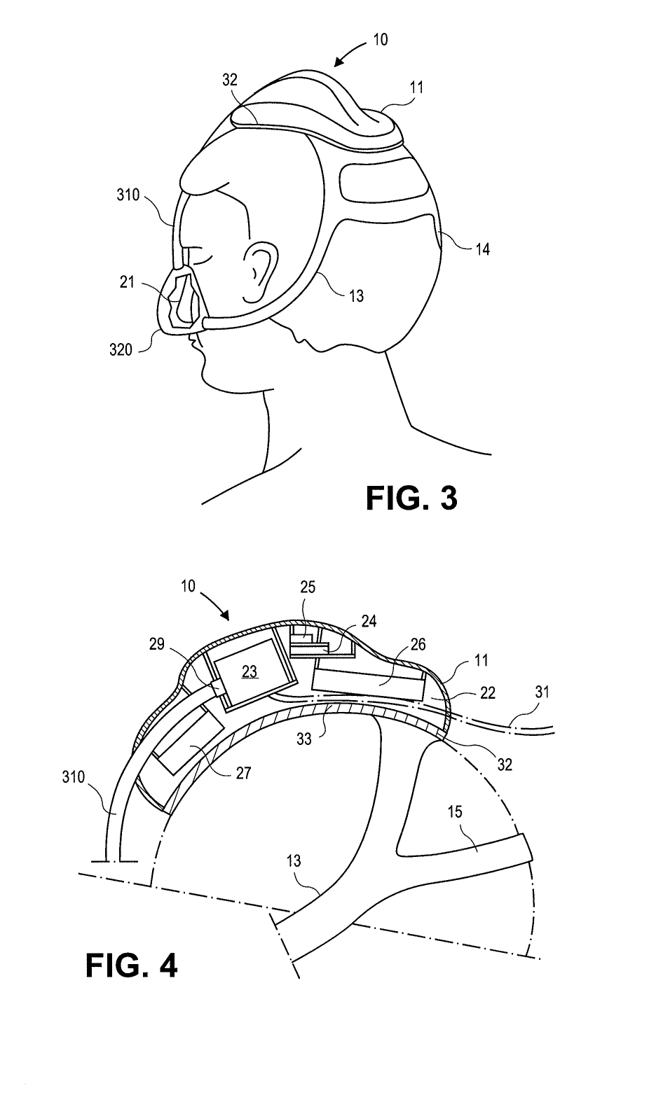

FIGS. 1-3 are perspective views of a PAP system 10 as described here. As shown, the system 10 may include a system housing 11 and a harness assembly 12 secured to the bottom of the housing. The harness assembly 12 has a plurality of straps 13 and 14 and cross strap 15 to secure the housing 11 to a patient's head 16 as shown in FIG. 2. The free ends of straps 13 and 14 have push in type connectors 17 and 18, as shown in FIG. 1, for securing the free ends to user interface 320 as shown in FIG. 2. The straps 13-15 hold a user interface 320 (e.g., a mask), as shown in FIG. 2, in a sealed engagement with the patient's nose 21.

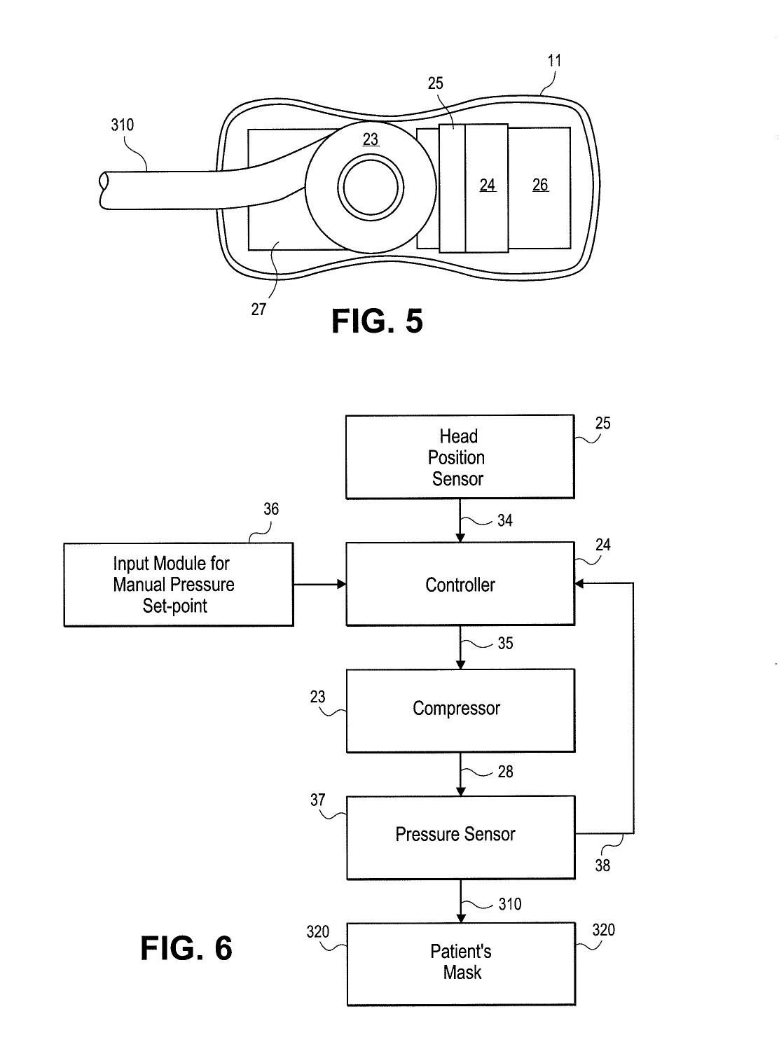

As best shown in FIGS. 4 and 5, the interior 22 of system housing 11 may contain a compressor 23, a controller 24, a position sensor 25 and batteries 26 and 27 to supply electrical power to the compressor 23. It should be appreciated that in some variations the system 10 may not include a position sensor. An air delivery matrix 310 may be secured to a discharge 29 of the compressor 23 (or another suitable pressure source) and may extend to the user interface 320 for delivery of a breathable gas to the patient's nose 21 as shown in FIG. 2. A system activation/calibration button 30 may be provided on the front of the housing 11, which may be configured to activate and/or calibrate the system 10 when pressed. An electrical power chord 31 (shown in phantom) may be provided for powering the compressor 23 directly from a separate electrical source such as an electrical outlet or battery pack (not shown) or for recharging one or more of batteries 26 and 27. A vibration and sound deadening layer 32 may be provided on the bottom 33 of the system housing 11 and between the compressor 23 and a supporting structure (not shown) supporting the compressor.

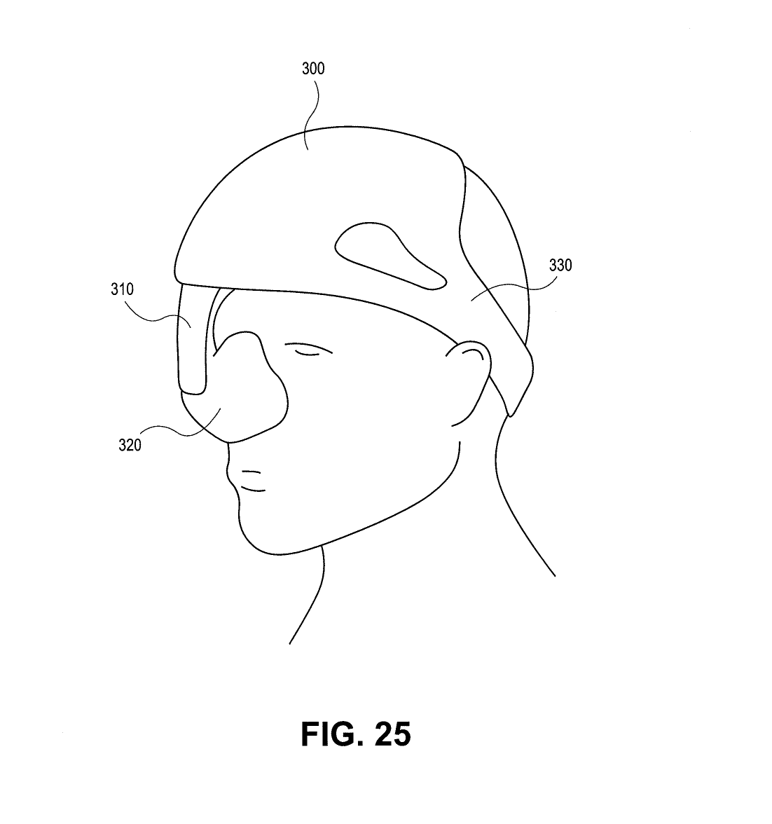

FIG. 25 shows a perspective view of one embodiment of a wearable CPAP system. As shown there, the system may include a system chassis 300, strap 330, air delivery matrix 310, and user interface 320. The air delivery matrix 310 may be situated between the system chassis 300 and the user interface 320. Pressurized air may pass from the system chassis 300 through the air delivery matrix 310 to the user interface 320, which may provide the pressurized air to the patient. Generally, the air delivery matrix comprises an elongate, member which may be configured to pass air through a non-hollow portion of the member. In some variations, the air delivery matrix comprises an outer matrix and an inner matrix, as will be described in more detail below. In some variations, the outer matrix may be formed from a different material or materials than the inner matrix. In other variations, the outer matrix may be formed from the same material as the inner matrix, but may have a different density than the inner matrix.

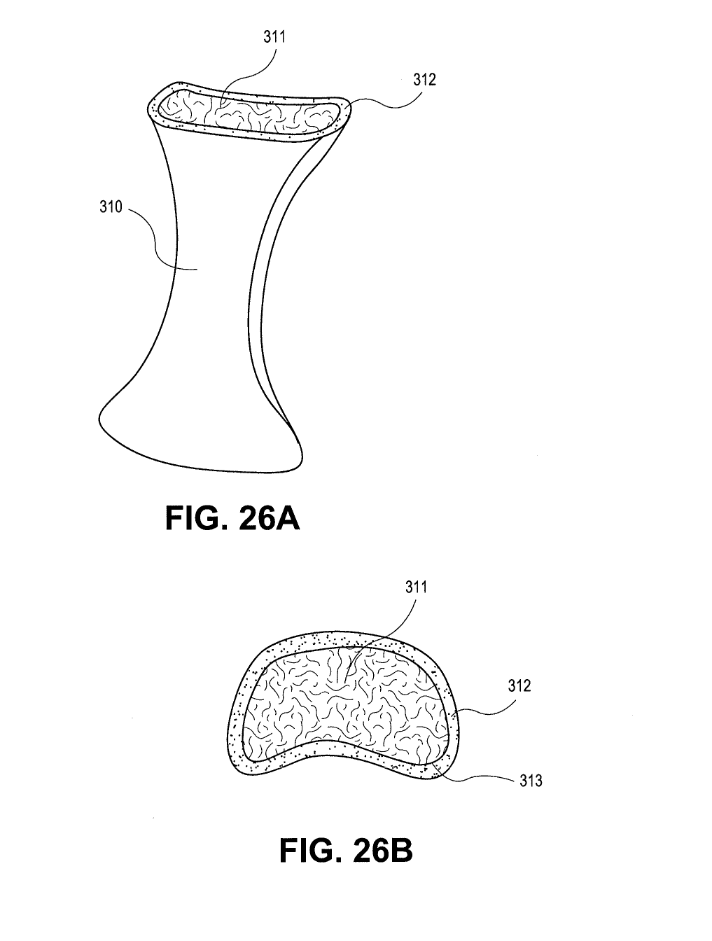

FIG. 26A shows a detailed view of the air delivery matrix 310. As shown there, the air delivery matrix 310 may comprise an outer matrix 312 which is filled an inner matrix 311. In some instances the outer matrix 312 may be tubular in shape. The inner matrix 311 and the outer matrix 312 may each be formed from one or more porous materials. The inner matrix material 311 generally has larger pores, or openings, compared to those of the outer matrix material 312, which may provide a lower resistance to airflow through the inner matrix 311 than the outer matrix. In some variations, the inner matrix material may be configured to allow airflow at typical PAP pressures and flow-rates without undue additional resistance. Such a resistance may fall in the range of 0.05 to 1.5 cm H₂O. The inner matrix material 311 could be comprised of a three dimensional arrangement of woven fibers, reticulated foams, open cell foams, polymers, elastomers, sponge materials, combinations thereof, or the like. In some variations, the inner matrix material 311 can be a reticulated foam or similar material with a “pores per inch” (ppi) rating in or near the range of 10-200 ppi. In some embodiments, the inner matrix material 311 may have a density in or near the range of 0.05 to 2.0 pounds per cubic foot (pcf) and an interior force deflection rating of 20-85. The inner matrix material 311 may allow the passage of air from a blower (not shown) inside the system chassis 300 to the user interface.

In some instances, the air delivery matrixes described here may be configured to provide humidification of the air that passes through the air delivery matrix 310. Generally, PAP systems that humidify the pressurized air produced by the system require a sizeable reservoir for storing distilled water, an element for vaporizing the water, and in some instances an element for heating the water. These humidification systems may be large, heavy, and energy- and maintenance-intensive, and thus may be impractical for providing humidification in PAP systems (especially wearable systems). In contrast, an air delivery matrix as described here may provide lightweight and energy-efficient humidification.

For example, in the variation of the air delivery matrix 310 shown in FIG. 26A, the inner matrix material 311 may be configured to provide humidification of the air passing through the inner matrix 311. When the user exhales, moisture inherent in the breath may be captured by the inner matrix material 311 as the exhaled air passes into the inner matrix 311. Upon inhalation, air passing into the user interface via the inner matrix 311 may be humidified by this moisture. Humidification can increase the comfort and compliance of CPAP users. Certain treatments or coatings are possible to enhance this property of the inner matrix material. For example, NaCl, other salts, chemicals and compounds may be used to coat the inner matrix material, and may increase the ability of the inner matrix material to capture moisture from moist air and to release the moisture into drier air. The exposed surface area of the pores of the inner matrix material can be adjusted to achieve the desired humidification performance. In some cases, having a longer, wider air delivery matrix will allow greater surface area of the inner matrix material, thereby enhancing the capture and re-delivery rate of moisture. Additionally, in some embodiments (not shown) the inner matrix may be pre-wetted with moisture in the form of a liquid or gel to assist in humidification. It should be appreciated that the air delivery matrixes described here may be used with either wearable or bedside-console-based CPAP systems to humidify air deliver to a patient's airway.

The air delivery matrixes described here may be configured to adjust one or more airflow patterns the through the air delivery matrix. In some variations, the inner matrix material 311 may allow for the adjustability of airflow patterns, through variations in its density and material. The cross-section of the air delivery matrix can be varied to achieve a desired airflow pattern and/or rate. It may be desirable to have portions of the inner matrix material wherein the air flows relatively faster to deliver the desired pressure and flow to the user. Conversely, it may be desired to have other portions of the inner matrix material wherein the air flows relatively slower to maximize the amount of moisture that may be captured by the air delivery matrix for re-delivery to the user. The airflow speed may be adjusted through the pore size of the inner matrix and/or the density of the inner matrix material may also be varied, either in whole or in portions or in cross-sectional regions, so as to achieve the desired performance. For instance, throughout the inner matrix material a gradient across the cross-section may be desired to allow optimal flow through the center, with slower flow toward the outside of the inner matrix. Conversely, there may be instances where it is desired to channel some or all of the flow to the outer portions of the inner matrix material, such as to enhance the humidification or flow characteristics.

The density of the inner matrix material 311 may be uniform, but it may also be varied. For example, the density or airflow characteristics can vary longitudinally along a length of the air delivery matrix. It may be desired to have the inner matrix material 311 more dense nearer to the user, enabling it to better capture the exhaled moisture. Further, it may be desired to have more than one zone of higher density along the path of the inner matrix material 311. It may be optimal to have a higher density at each end of the air delivery matrix 310. The density may also be varied radially. The density can be highest at the edges of the air delivery matrix 310, close to the outer matrix material 312. This would allow brisk airflow through the center of the air delivery matrix 310, and could limit turbulence at the edges. Reduction of turbulence may reduce noise that may occur in the air delivery matrix 310. The inner matrix material 311 and the outer matrix material 312 may be configured to perform noise dampening. In another embodiment, the inner matrix material may be absent in portions of the air delivery matrix. For instance, the air delivery matrix may begin with a section lacking the inner matrix material, and then include the inner matrix material closer to the user interface (not shown).

In PAP systems, air may be compressed to achieve a desired pressure and flow rate. At this increased pressure and flow rate, the air may feel cooler to the user, and causes an increased rate of evaporation of moisture within the air path and airway and consequently, cooling. To counteract this effect, it may be desirable to heat the air provided by system. In wearable systems, however, it may be undesirable to incorporate a heating element into the system, as a heating element may be heavy and energy intensive, and may also pose a safety hazard when positioned on the head of a patient. Accordingly, in some variations the air delivery matrixes described here may be configured to regulate the temperature of pressurized air delivered to the patient.

For example, in some variations the inner matrix material may also be configured to regulate the temperature of the pressurized air that is delivered. The inner matrix material helps to capture the heat of the breath upon exhalation, and re-deliver that heat during inhalation. For example, the inner matrix may comprise one or more materials with low specific heat capacity, such as polymers, elastomers, ceramics, certain metals and composites, combinations thereof and the like. Upon exhalation, heat in the exhaled air is absorbed by the inner matrix material 311. Upon inhalation, this heat may be transferred to pressurized air that flows through the inner matrix material 311, which may thereby increase the temperature of the air passing into the user's airway. The inner matrix material can be constructed or coated with material that enhances these heat storage and/or transfer properties. For example, material which readily heats and cools could be used to maximize the heat transfer. Materials or coatings with good heat conductivity can be employed for this purpose. In some instances, additional materials with high specific heat capacity may be incorporated into the air delivery matrix for heat storage.

In some instances, the air delivery matrix may also dampen noise that may be created by air movement from a pressure source to the patient's airway. For example, in some variations the inner matrix materials described herein may reduce turbulence of air flowing through the air delivery matrix, and thus may reduce the noise caused by the airflow. The inner matrix material may be configured to allow for a smoother, more even flow of air. For example, inner matrix material may dampen the ability of the airflow to accelerate and decelerate in various parts of the airflow circuit. This more even airflow can create less noise, which may offer a significant advantage over existing tubing-based systems.

FIG. 26B shows a cross-section of the air delivery matrix 310. The outer matrix material 312 can be comprised of a three dimensional arrangement of woven fibers, reticulated foams, open cell foams, polymers, elastomers, sponge, or similar materials. The outer matrix material 312 is higher in density and has a smaller pore size than the inner matrix material 311, and may be configured to allow a significantly lower flow rate through the outer matrix than through the inner matrix. The material density, pore size, raw material, and thickness of the outer matrix may be varied to achieve the desired airflow resistance. In some cases, the outer matrix may be configured to such that air does not flow through the outer matrix material. In some cases, it selectively allows airflow in certain areas through variations in its density, or through openings positioned on its surface. In some instances, the outer matrix material 312 may be configured to provide for an exchange of air between the inner matrix material 311 and atmosphere. The may facilitate the removal of exhaled gases such as carbon dioxide from the inner matrix. This air exchange through the outer matrix material can serve to allow the exit of exhaled gases such as CO₂. The exchange of air through the outer matrix material can be designed to occur along its entire length and surface area. Alternatively, this exchange may be allowed only in certain areas, and can be varied longitudinally, radially, or some combination thereof. For instance, it may be desired to avoid the egress of gases in the direction of the user (e.g. when air is flowing through the air delivery matrix from a pressure source to a user interface). It may be desirable to allow the passage of exhaled gases in a focal region closer to the user. It may be desired to have air exit more easily (i.e. a more porous matrix or outright openings) close to where the matrix connects to the output of the flow generator, helping to assure the air going to and coming from the patient passes through a significant portion of the vapor/heat recovery matrix. The rate of exchange of these exhaled gases through the outer matrix material can be controlled through changes in material, pore size, density, geometry and other methods.

The outer matrix material 312 may provide structure support to the air delivery matrix 310, and may help hold air delivery matrix 310 in place and maintain a comfortable fit and good seal on the user interface 320. This structure and selective rigidity can be achieved through the use of materials which provide the adequate flexibility to keep the user interface securely in place and avoid leaks. Further, the outer matrix material may incorporate the placement of one or more additional support members to provide rigidity to the air delivery device. These support members can be made of metal, plastic, composites or similar materials. These structural members can be flexible so as to be positioned as desired and allow adjustability for fit with various users, while also providing enough rigidity to maintain their adjusted position and help prevent leaks. In some variations, the support member may be shapeable by a health care professional or user, which may allow the individual to set the support member to a desired shape. In one embodiment, shape memory metals may be used for at least a portion of these support members to provide support to the air delivery matrix. The outer matrix material may be formed in manufacturing around these support members, or the outer matrix material may be formed with cavities and openings allowing for the placement of these structures later in manufacturing or assembly. Additionally, the support members may be connected to one or more of the system chassis, pressure source, and user interface. The air delivery matrix may be configured to slide off of or otherwise disengage the support members to allow for cleaning or replacement of the air delivery matrix.

The outer matrix material 312 can also provide a channel or lumen to the user interface 320 to allow for measurement of the pressure in the user interface 320. These measured pressures can be used by a PAP control unit to inform the control and adjustment of the airflow generator to maintain the desired, optimal pressure for the user at each point in the respiratory cycle.

In some variations, the inner matrix may comprise one or more channels or lumens for placement of a support member therein. Positioning a support member in the inner matrix may allow the support member to provide structural support, but may also allow the external portions of the air delivery matrix to be soft or pliable. In some variations, both the inner matrix and the outer matrix may include one or more lumens.

As shown in FIG. 26B, the air delivery matrix optionally, includes a separation layer 313 between the inner and outer matrix materials. Example separation layers 313 include an adhesive or thermally altered layer to bond the inner and outer materials together. A separation layer 313 may include a thin film. In one embodiment, this thin film could be porous or gas permeable in nature. In another embodiment, the thin film may be non-permeable. It could likewise be a combination of permeable and non-permeable in various regions to enhance the functionality. For example, the thin film layer may be non-permeable for the majority of its length, but permeable near the user interface or near the system chassis to allow the exchange of exhaled gases such as CO₂. Likewise, the permeable area may be arranged to point out, away from the user, to reduce the likelihood that the air outflow disrupts the user. Further, the permeable area may be arranged to direct air in a diffuse manner, such that it avoids the likelihood that the air outflow disturbs a bed partner.

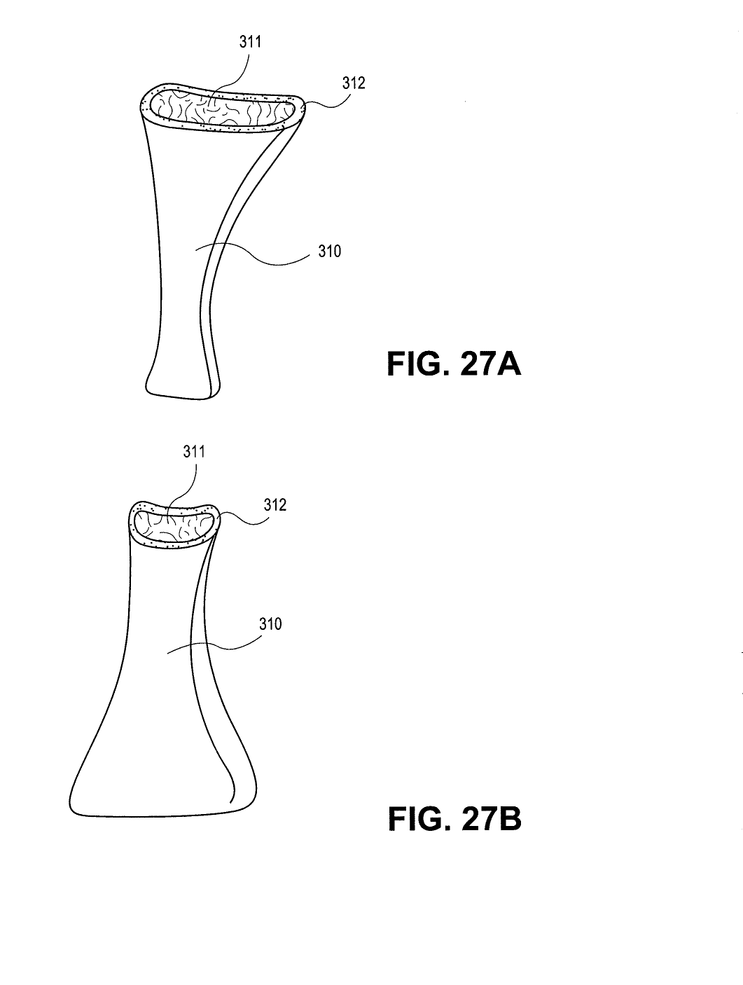

FIG. 27A shows the air delivery matrix 310, which tapers from the top (near the system chassis), narrowing at the bottom (near the user interface). This geometry would provide for the acceleration of air near the user interface 320, and slower airflow near the system chassis 300. Where the airflow is slower, the exchange of heat and moisture may be enhanced.

FIG. 27B shows the air delivery matrix 310, which tapers from the bottom (near the user interface), narrowing at the top (near the system chassis). This geometry would provide for the acceleration of air near the system chassis 300, and slower airflow near the user interface 320. This configuration could allow greater capture of heat and moisture closer to the user. It could also ease the transition from the narrow exit of the system chassis 300 to the broader volume of the user interface 320.

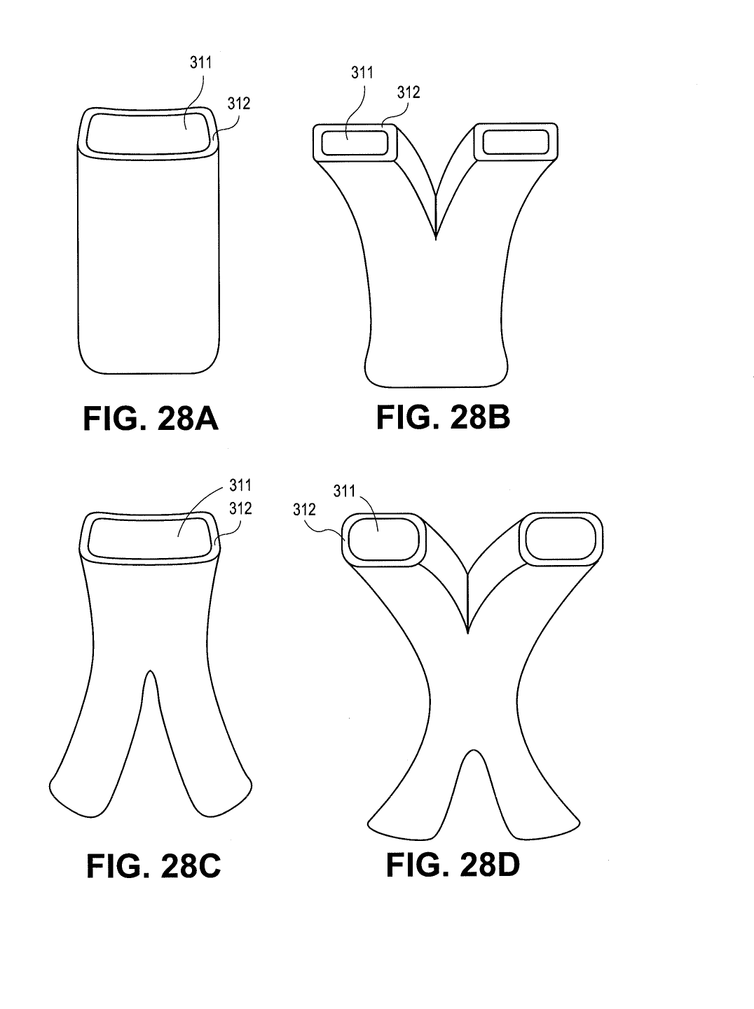

FIG. 28A shows an air delivery matrix 310 that is largely uniform in width throughout its length, which may provide a uniform flow rate through the air delivery matrix. It should be appreciated that if the cross-sectional area of the air delivery matrix 310 remains the same, the geometry of the air delivery matrix may be altered while still maintaining a given flow rate.

FIG. 28B shows an air delivery matrix 310 that begins as two separate portions but merges into one on the user interface 320 side. This design could offer compensatory flow rates through the two portions, which may be of benefit in certain sleeping positions.

FIG. 28C shows an air delivery matrix 310 that begins as one portion but splits into two portions on the user interface 320 side. This design could offer a geometry that may be well suited for integration with the user interface 320, as well as benefits in certain sleeping positions such as the lateral position.

FIG. 28D shows an air delivery matrix 310 that begins as two separate portions but merges into one in the mid section and then again splits into two separate portions on the user interface 320 side. This design could offer ergonomic, flow, and aesthetic benefits.

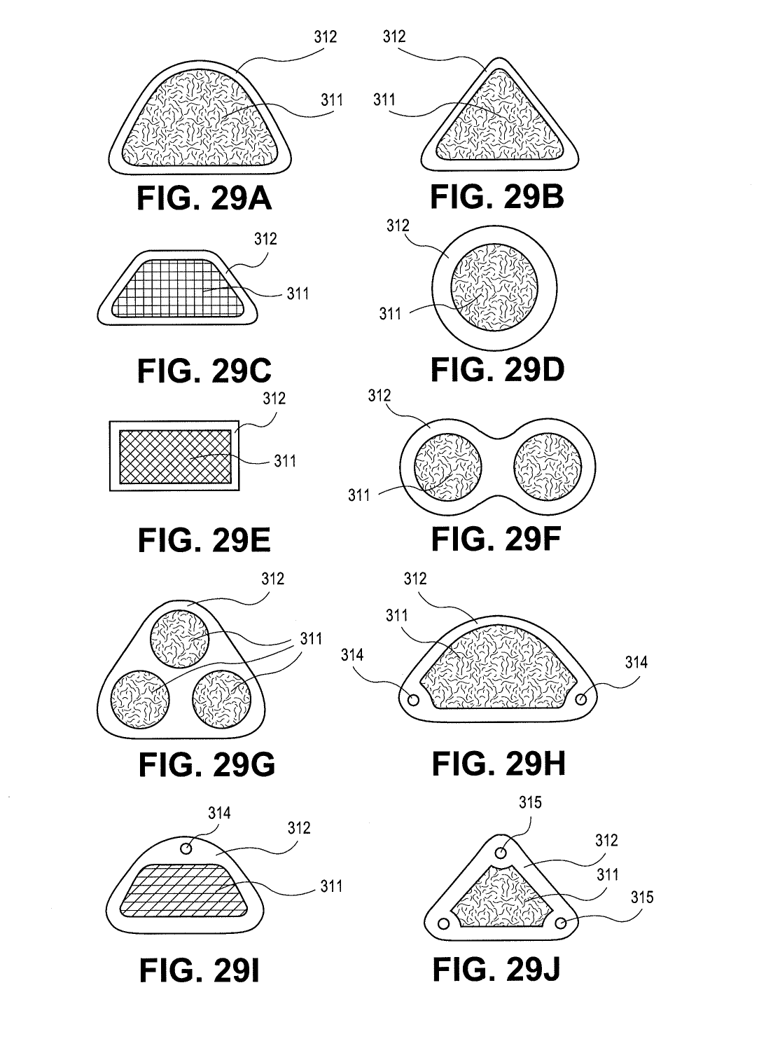

FIGS. 29A-29J show cross-sectional views of the air delivery matrix 310. Any of these arrangements may offer ergonomic or aesthetic benefits to the user. FIG. 29A shows a semi-circular geometry which may relate well to the face of the user. FIG. 29B shows a largely triangular geometry, which may provide structural strength to resist unwanted bending and offers aesthetic benefits. FIG. 29C shows a largely trapezoidal geometry which allows adequate airflow in a smaller profile. FIG. 29D shows a largely circular geometry, which may maximize the ratio of the flow area to external flow circumference, which helps flow efficiency and reduces drag. FIG. 29E shows a largely rectangular geometry which could help keep the air delivery matrix 310 positioned close to the face of the user.

FIG. 29F shows an embodiment with two inner matrix material portions surrounded by the outer matrix material 312. This arrangement provides multiple airflows in a low profile. FIG. 29G shows an embodiment with three inner matrix material portions surrounded by the outer matrix material 312. This arrangement may allow increased structural support of the air delivery matrix 310. FIG. 29H shows an embodiment with two lumens 314 incorporated into the outer matrix material 312. These lumens can be used to measure the pressure in the user interface 320. Accurate and rapid pressure measurement improves the responsiveness of the PAP system and contributes to improved performance, comfort, and ultimately, compliance. In other instances, one or more support members (such as discussed above) may be inserted into one or more of the lumens. FIG. 29I shows an embodiment with one lumen 314 in the outer matrix material 312 for the sampling of pressure in the user interface 320. FIG. 29J shows an embodiment with three lumens 315 incorporated into the outer matrix material 312. These lumens 315 can be used for either pressure measurement or for the insertion of support members into the air delivery matrix 310 to hold it in place and secure the user interface 320 as desired.

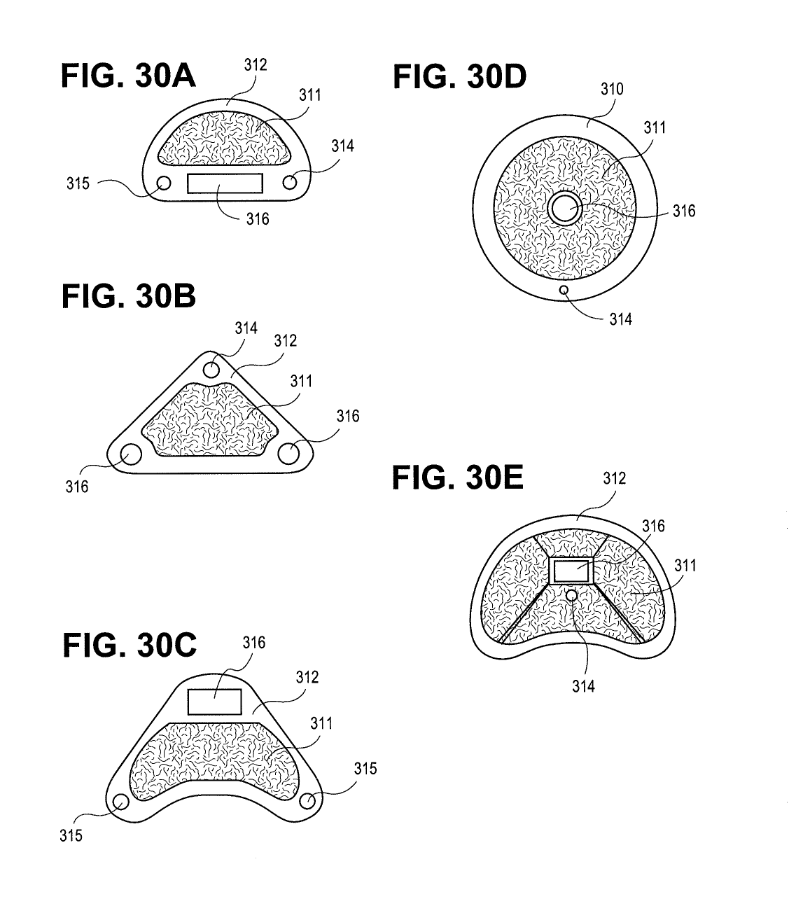

FIG. 30A shows an embodiment with three lumens 314, 315, 316 incorporated into an outer matrix material 312 having a semi-circular shape. FIG. 30B shows an embodiment with three lumens 314, 316 incorporated into an outer matrix material 312 having a triangular shape. FIG. 30C shows an embodiment with three lumens 315, 316 incorporated into an outer matrix material 312 having an arrowhead shape. The lumens of these variations can be used for either pressure measurement or for the insertion of structural elements, or both. In some instances, it may be advantageous to have the structural elements pass through a lumen on the outside of the air delivery matrix 310, further from the user's face. In other cases, it may be desired to have the structural elements pass closest to the user's face. FIG. 30D shows another embodiment with a lumen 316 down the center of the air delivery matrix, which may be configured for placement of a support member therein. In some instances, the lumen may extend through the inner matrix material. Alternatively, a support member could be integrated directly into the inner matrix material. The support member may be positioned and adjusted to achieve comfort and fit. Also shown in FIG. 30D is a lumen 314 configured to monitor pressure, which may pass through the outer matrix material. FIG. 30E shows another embodiment wherein lumen 316 is rectangular in cross section, which may allow for placement of a rectangular support member. This shape may provide more stability in one direction (e.g., in a lateral direction) while providing flexibility in a second direction (e.g., in the sagittal plane). Also shown in FIG. 30E is a lumen 314 for monitoring pressure. As shown there, the lumen 314 may extend through the inner matrix material, although in other instances it may extend through the outer matrix material such as shown in FIG. 30D.

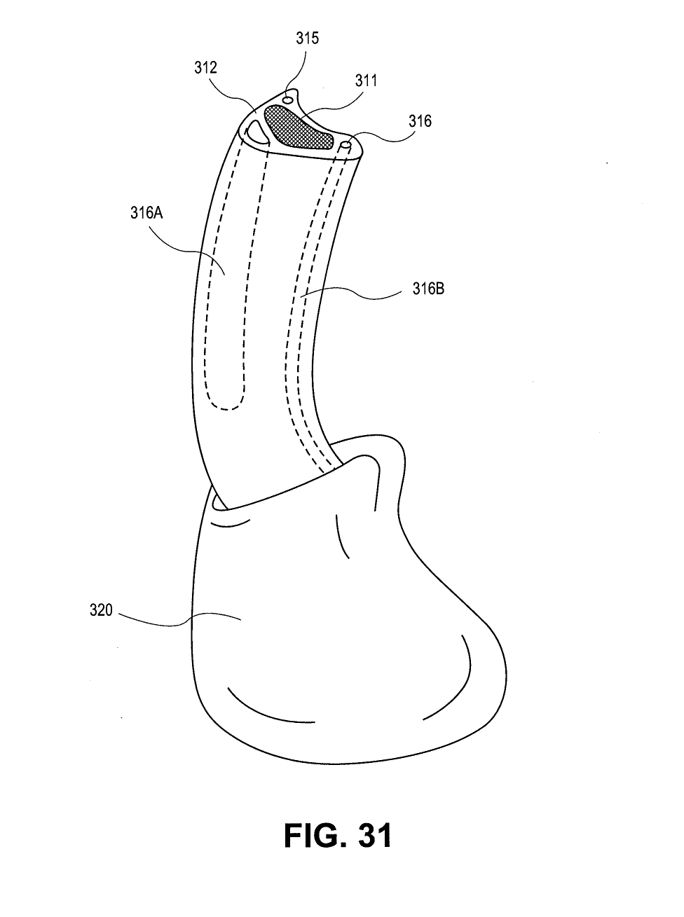

FIG. 31 shows a portion of the air delivery matrix 310 and the user interface 320. In this figure, the outside lumen 316A only passes through a portion of the air delivery matrix 310, not the entire length. This may be desired to help position the user interface 320. Various other combinations of the lumens can be made to extend only partially through the length of the air delivery matrix 310 to provide support for a specified portion of the air delivery matrix.

These various arrangements can offer benefits such as better forming to the face or head of the user, better adapted to head structures, more aesthetically appealing, or more comfortable in various sleeping positions.

### System Chassis

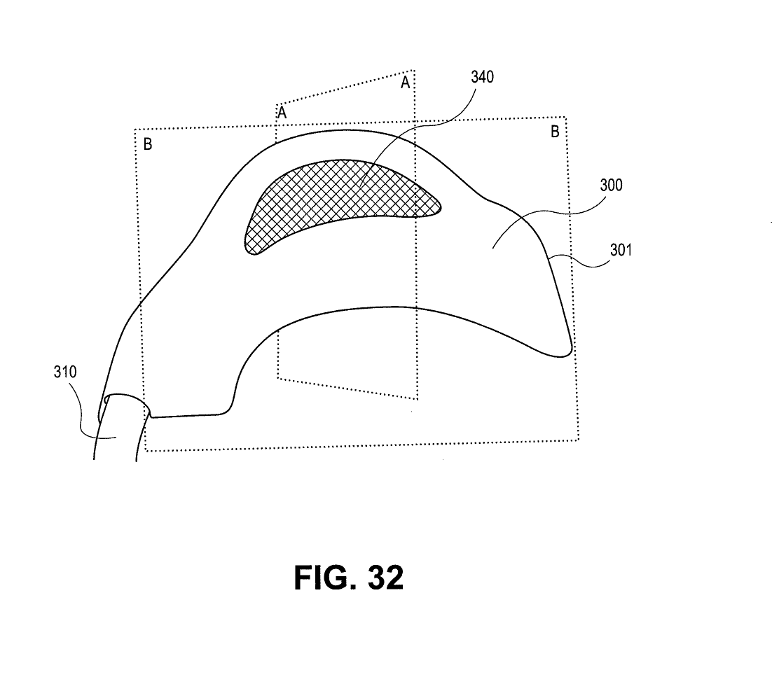

FIG. 32 shows an embodiment of a system chassis 300 that may be used with the systems described here. The chassis 300 may be configured to be worn on a patient's head. In some instances, the system chassis material may comprise a foam that may be used to secure the elements of the wearable CPAP system in place, and define certain structures, such as plenums, within the system. Forming a chassis of a CPAP system at least partially from foam may reduce the weight of the chassis while still providing structural support and protection (e.g., protection against impact) to the internal components of the chassis. In some variations, the foam may be engineered to absorb both vibration and sound, which may enhance user comfort (as well as reduce distractions provided to individuals near the user). Additionally, a foam chassis may insulate the user from heat generated by the system. Additionally, foam chassis may be comfortable when worn by a user (e.g., when compared to a housing formed from a rigid plastic), which may reduce the likelihood that a chassis worn during sleep will disturb a user's sleep.

FIG. 32 shows a perspective view of a portion of the system chassis 300 including an air inlet filter 340. The air inlet filters 340 are pictured in one possible location, but may be located in any suitable portion of the chassis. These filters can be replaceable or washable at regular service intervals. Also shown is one way in which the air delivery matrix 310 can interface with the system chassis 300. The figure also indicates two cross-sectional planes for further detail, A-A and B-B.

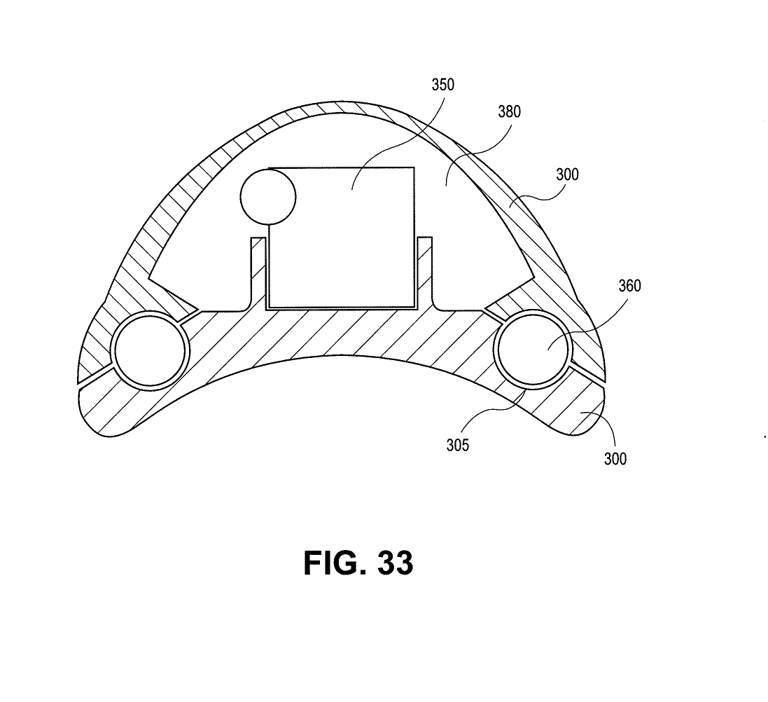

FIG. 33 shows a cross section of one variation of the chassis 300 taken along the A-A plane shown in FIG. 32. It illustrates one way in which two halves, or multiple parts of the system chassis 300 could mate together to secure the system components in place. Multiple parts of the chassis could alternatively come together from the side to form the chassis. In addition to securing the components like the blower 350 and batteries 360, the system chassis forms spaces for the air path, such as the air plenum 380.

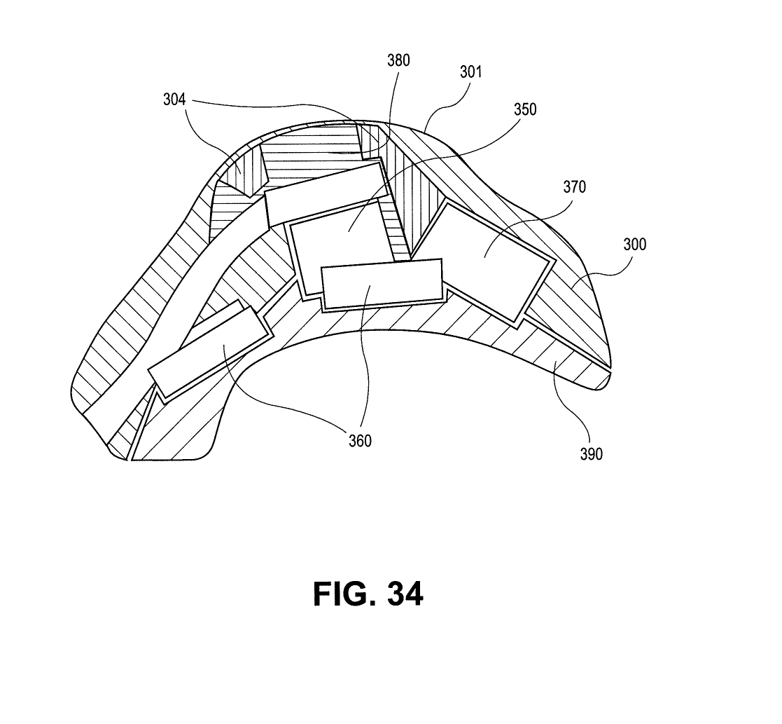

FIG. 34 shows a longitudinal cross section taken along the B-B plane. It illustrates a possible arrangement of the components of the system within the system chassis 300. The blower 350, batteries 360, and control electronics 370 are held securely in place. The system chassis 300 can be made from a number of materials such as foams, polymers, elastomers, injection molded foams, injection molded polymers, injection molded elastomers, thermal processed foams, polymers, or elastomers. In one preferred embodiment, the system chassis 300 is comprised of injection molded expanded polypropylene, which may reduce the weight of the chassis and provide impact absorption as discussed above. Further, these materials are proficient at absorbing sound vibration and heat, thereby reducing the discomfort of the user. Finally, these materials may be comfortable to be worn on the head, and may flex and give with the movement of the head during sleep. They may also be aesthetically appropriate for the sleeping environment.

In some instances, a cloth or fabric 301 covers the system chassis 300. This may provide for a more comfortable device. It also allows for easier customization of color, pattern, and style, which may provide more choices to a user in selecting the appearance of the chassis. This increase in control of the device aesthetics may increase patient compliance.

An interface material 390 is also shown in FIG. 34. It can be made of a softer material for enhanced comfort and fit.

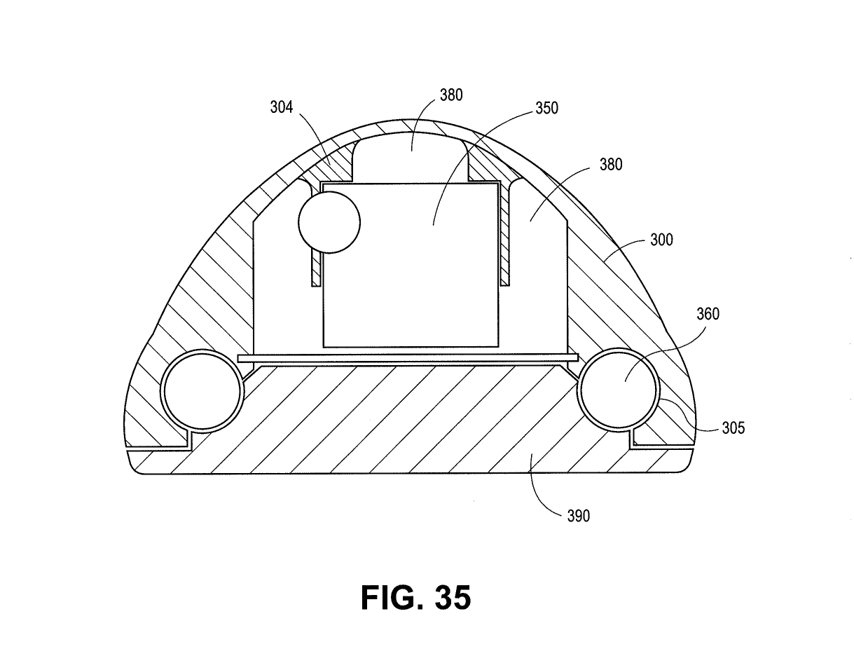

FIG. 35 shows a cross section of a portion of the system. Here the blower 350 may be secured by capturing elements 304 of the system chassis 300. The system chassis 300 also may create the space for the air plenum 380, and may capture the batteries 360 in press fit cavities 305. As shown there, the blower may be suspended above the head, which may reduce the transfer of vibrations to the user. The press fit construction partially or completely eliminates the need for fasteners which may be expensive, heavy, and add cost in labor and potential for error and malfunction during service life. The interface material 390 may complement the system chassis 300 in form, and may provide a softer material for comfort of the user.

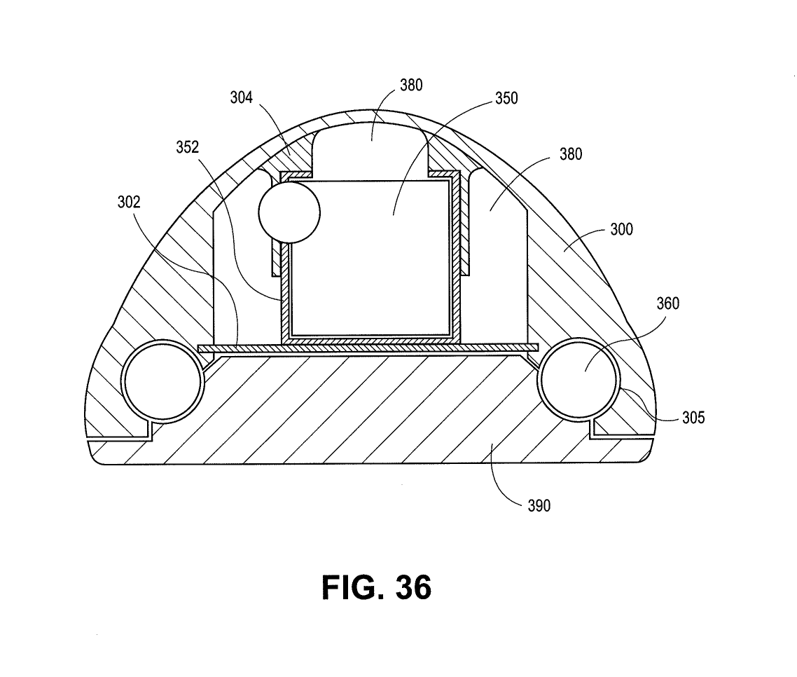

FIG. 36 shows another embodiment in cross section. This embodiment includes a blower suspension element 352 which can be made out of a foam, polymer or elastomer. In one preferred embodiment, the blower suspension element 352 may be made from a silicone elastomer. The material may be chosen to suspend the blower 350, hold it securely in place, help protect it from impact or jolting, help reduce the transfer of any vibrations it might produce, and thereby reduce the noise of the system. Further shown is a chassis strengthening member 302 which helps to define the air plenum 380 space and to provide further structure for the system chassis 300. Mating with the system chassis 300 is the interface material 390. It can be made of a foam, sponge, polymer, elastomer, composite, or contain a gas or gel for comfort.

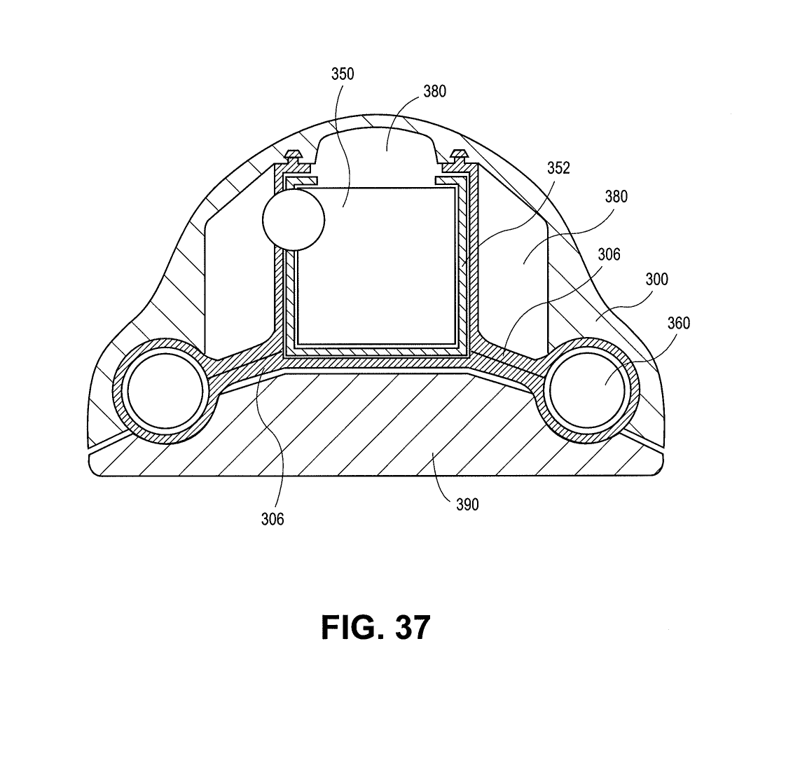

In another preferred embodiment shown in FIG. 37, the blower 350 may be held by a blower suspension element 352, which may then be held by an inner chassis 306. The inner chassis 306 can be made of polymer, elastomer, or composites. The inner chassis 306 may serve to provide a relatively rigid, secure arrangement of the components. The inner chassis 306 mates with the system chassis 300. The interface material 390 may be flat-bottomed and nest with the inner chassis 306 and system chassis 300. The flat bottom may allow the device to rest comfortably on flat surfaces when not in use. It also may provide additional cushioning material without expanding the outer overall dimensions of the device. The addition of the inner chassis 306 may allow the system chassis 300 to become more flexible and less rigid, as the required rigidity can be provided by the inner chassis 306.

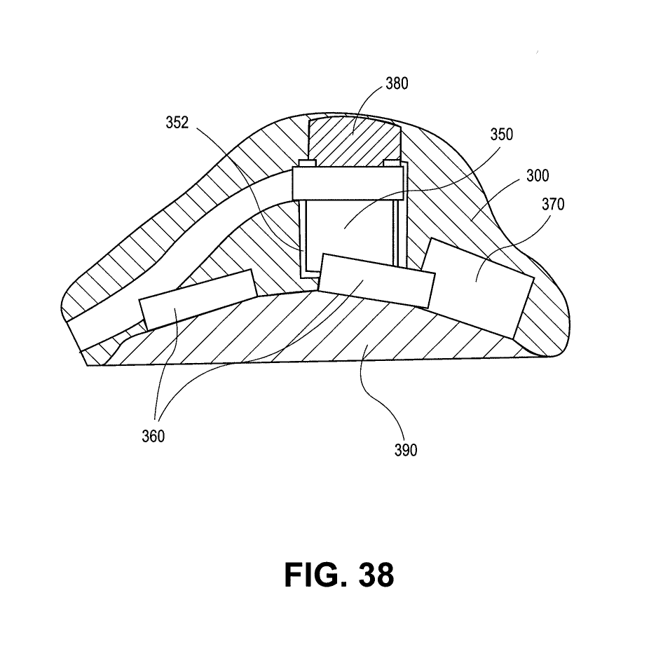

FIG. 38 shows a longitudinal cross section of an embodiment of the device. The blower 350, blower suspension element 352, batteries 360, control electronics 370 are all captured and held by the system chassis 300. Below the system chassis 300 is the interface material 390. It may be flat-bottomed and designed to interface between the user and the system chassis 300.

The foam structures described herein could also be realized using other expanded polymers and elastomers. The foam structure provides a further advantage in terms of cost and manufacturing. The foams may be formed into complex shapes, and the cost of each piece may be relatively low. Further advantages may be realized during manufacturing, as a foam can be formed to provide cavities for the snug, accurate fit of the PAP internal components. This may avoid the need for additional fasteners, which may introduce cost and/or the potential for manufacturing error or malfunction during service life.

### Inlet Filter

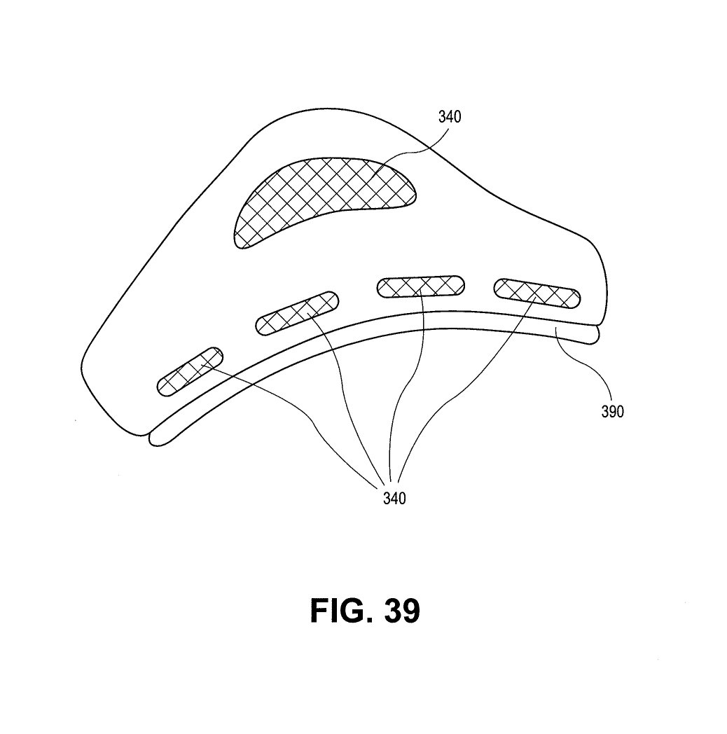

FIG. 39 shows an embodiment of system chassis comprising multiple potential placements of the air inlets and the air inlet filters 340. Additionally, the interface material 390 is shown extending beneath the lower surface of the system chassis 300. The interface material 390 could be comprised at least partially of an air bladder 391.

The inlet filter may prevent certain materials from entering the plenum or the airflow generator. The inlet filter material may include foam, such as reticulated foam, cloth, and/or woven fiber. The inlet filter can be replaceable periodically. The inlet filter can be washable. The filter may be configured to prevent dust, particulate, hair, fibers and the like from entering the inlet pathway and the flow generator. This may help maintain the flow generator and reduce the likelihood that such materials may adversely affect its performance. Further, such an inlet filter will help prevent these materials from being passed to the user's airway. An inlet filter may be especially important on a wearable PAP system because the air inlet on a wearable system may be positioned in close proximity to the body and/or bedding materials, elements of which may be drawn to the air inlet during operation of the pressure source. Optionally, the cloth covering of the foam structure containing the element of the PAP device could also perform a second function as an inlet filter.

### Air Bladder

FIG. 39 shows an embodiment of system chassis comprising an interface material 390 shown extending beneath the lower surface of the system chassis 300. The interface material 390 could be comprised at least partially of an air bladder 391.

The system described herein incorporates a cushioning element between the user and the device. The cushioning element is designed to make the user more comfortable by softening the contact area with the device and to improve the fit by allowing for varying sizes and shapes among the population of users. It increases user comfort by dampening vibration from the system. It also can help with dampening the noise created by the system. This cushioning element can be made from materials such as foam, expanded plastics, fabrics, synthetic or natural rubbers and the like.

In some variations, the system may comprise an air bladder 391. The air bladder may be positioned between the housing or chassis of the system when worn by a user. An air bladder may increase the comfort of the user, and may do so without significantly adding to the weight of the system (e.g., as the air bladder may be filled with air). Although typically filled with air, the air bladder may in some instances be filled with one or more liquids, gels. In some instances, the air bladder may house one or more foam members. In some instances, air bladder may distance the housing or chassis from the scalp of the user, which may reduce the amount of heat that may be transferred from the housing or chassis to the scalp while allowing heat to be effectively transferred away from the scalp to the surrounding air. Further, the air bladder 391 may offer a flexible fit for comfort and adjustability. The air bladder 391 does not have to be shaped to conform to a specific head shape. Instead, the plasticity of the air bladder 391 may allow it to comfortably fit on a range of shapes. The air bladder 391 might be selectively inflated and deflated by the user to achieve a desired fit. The air bladder 391 can be manufactured by selectively bonding two layers of substrate together to create a seal around their perimeter and to form geometry within the perimeter. This seal can be manufactured using heat, pressure, adhesives, or a combination thereof. In some variations, the air bladder 391 may be flat or near flat when deflated, which may facilitate placement of the system on a flat surface (such as a night stand) when not worn by the user.

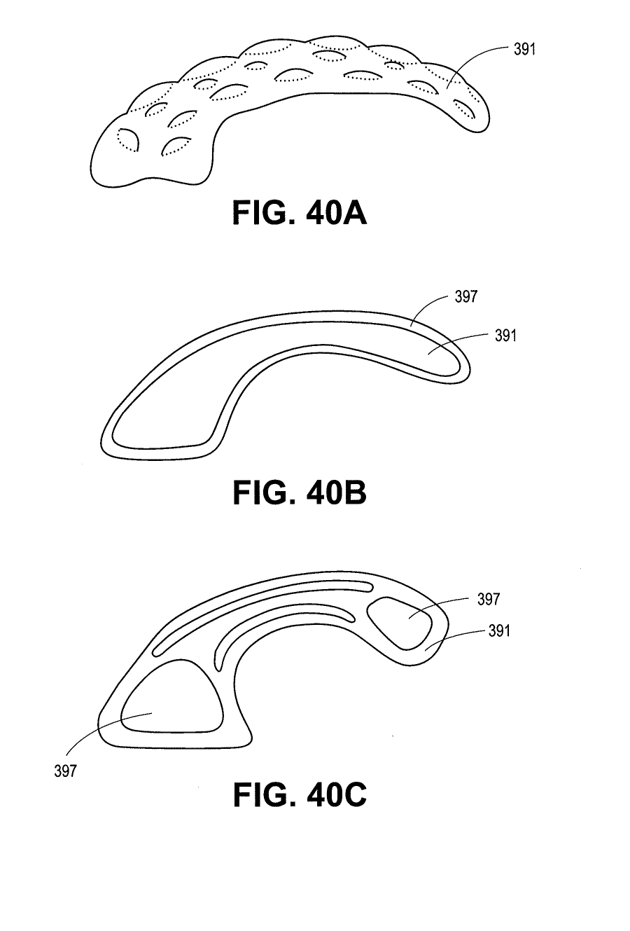

FIG. 40A shows an embodiment of the air bladder 391, comprising a series of three dimensional bumps on its surface. These bumps can be on one or both the top and bottom of the air bladder 391.

FIG. 40B shows a preferred embodiment of a cushioning element comprising an air bladder 391. The air bladder includes an air chamber 397 around the perimeter of the air bladder for added comfort and fit.

FIG. 40C shows an embodiment of the air bladder 391, comprising multiple air chambers 397. A chamber 397 at each end of the device provides fit and adjustability to varying head sizes and shapes. Other chambers 397 following the length of the air bladder 391 provide some structure as well as comfort and fit.

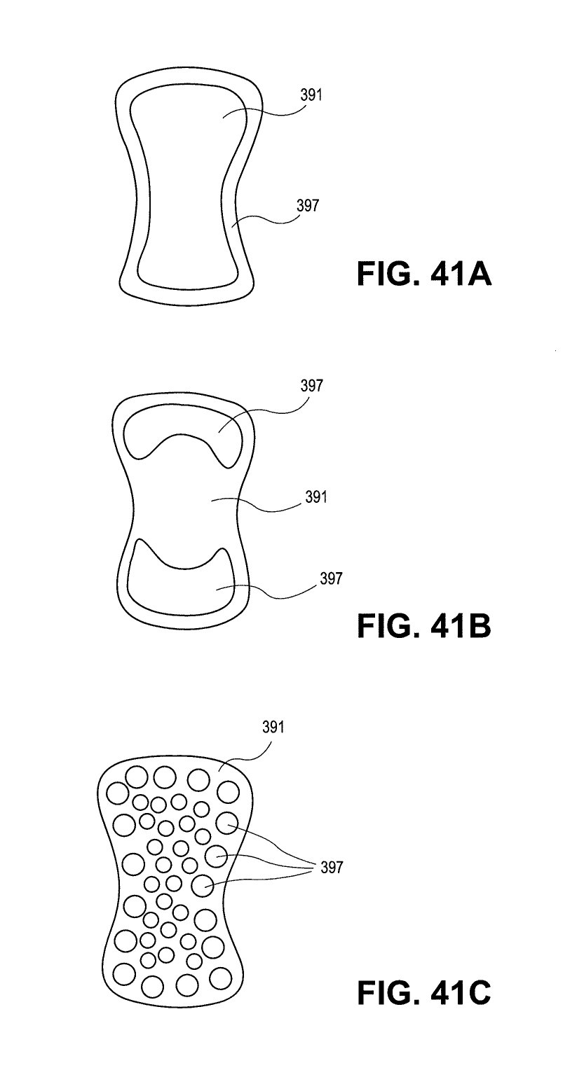

FIG. 41A is a top view of an air bladder 391 with an air chamber 397 around its perimeter.

FIG. 41B is a top view of an air bladder 391 with two air chambers 397, one at either end of the air bladder 391 for custom fit and comfort with the device.

FIG. 41C is a top view of an air bladder 391 with multiple air chambers 397, and three dimensional elements which help increase the surface area and heat transfer and ventilation capabilities of the air bladder 391.

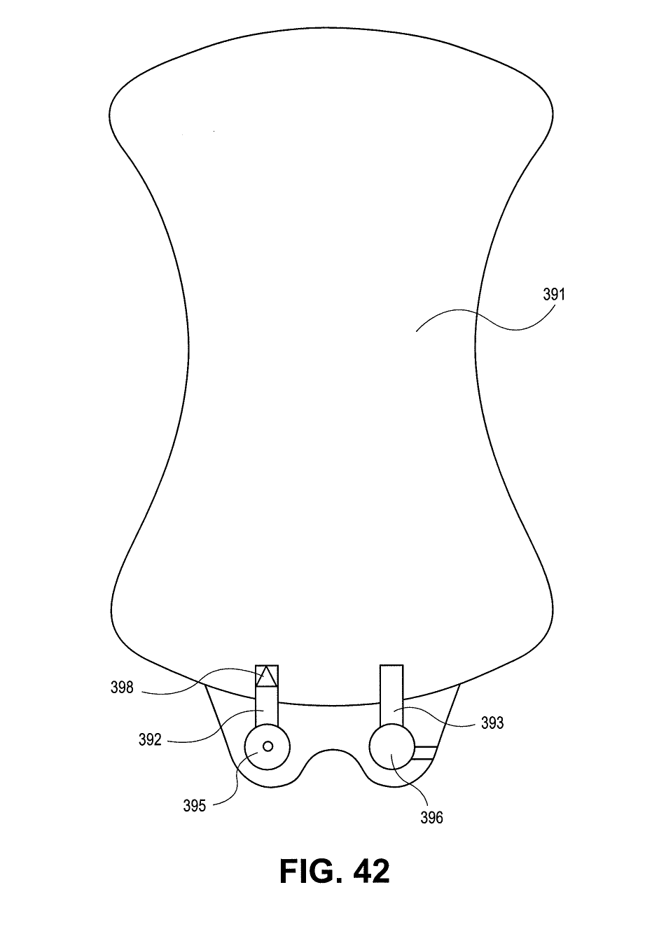

FIG. 42 shows a top view of an air bladder 391 with a manual mechanism for inflating and deflating the air bladder. In a preferred embodiment, the cushioning element comprises an air bladder which can be selectively filled with air to varying volumes and pressures to enhance fit and comfort. This air-filled chamber offers several distinct advantages. First, it provides excellent heat transfer. The lower surface of the air chamber can be designed to easily allow the transfer of heat to the air contained inside. The air inside allows quick distribution of the heat. It behaves more like the air surrounding a user's head, when the user is not wearing anything. This efficient transfer of heat from the device contact zone greatly enhances user comfort. It is proposed that any increase in user comfort can lead to increased use which directly benefits the user's immediate and long term health. The amount of air in the chamber can be adjusted by the user. A one-way valve system may allow a user to pump air into the chamber to fill it. More or less air may be desired to achieve a desired fit for the user. A second valve may allow the user to express air out of the chamber. This valving system may allow the user to adjust the air chamber fill for fit and comfort, and also for transport. When transporting the device, such as during travel, the air chamber can be emptied to reduce the amount of space the PAP device occupies, which may be advantageous during travel when bulkier systems may be difficult to transport.

The valve system for the chamber can be achieved at low cost using one way valves and release valves. The air chamber can be made from thin plastic sheets which are heat bonded together. Other construction techniques could certainly be used to achieve similar effect. This air filled chamber can be made to be disposable. As it sits directly adjacent to the user, it may be desired that it be replaceable with some frequency. This could be once per week, once every three months, or another such frequency.

As shown in FIG. 42, when the manual inflator 395 is squeezed, a small amount of air may be forced through the air bladder inlet 392, through the one-way valve 398 and into the air bladder 391. When this is done repeatedly, the air bladder 391 may be inflated stepwise to various inflation levels. The user can control and adjust the inflation of the air bladder 391 to achieve a desired comfort and fit. When the manual deflator 396 is squeezed, a relief valve is opened and air is allowed to escape the air bladder 391 and pass through the air bladder outlet 393 and into the atmosphere. In this way, the user can adjustably control the degree of inflation of the air bladder 391. This manual system can be constructed and low cost. It can be manufactured at such a cost so as to allow it to be replaceable every three months, or more frequently, as many patient contacting CPAP materials are typically replaced.

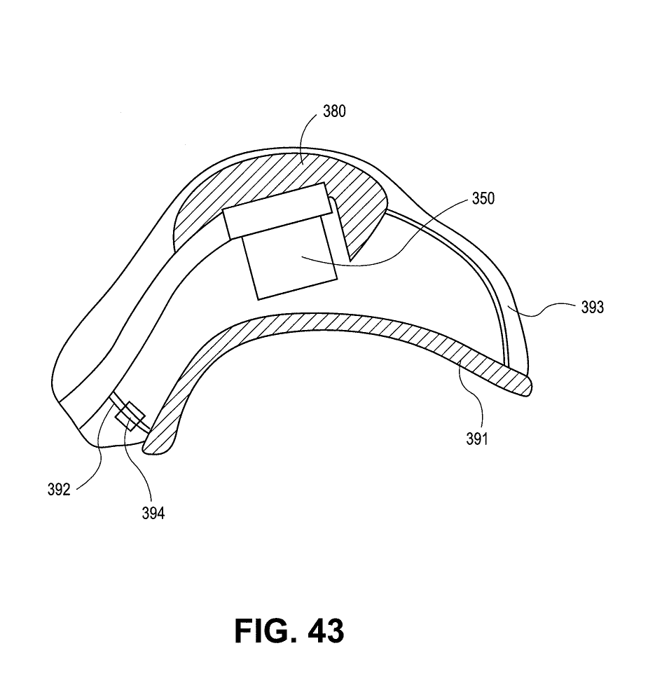

FIG. 43 shows another embodiment of an adjustable air bladder 391. In this embodiment, a portion of the air exiting the blower 350 may be channeled through the air bladder inlet 392, and through an optional valve 394 into the air bladder 391. The valve 394 can be static or adjustable in terms of cracking pressure and flow rate. After passing through the air bladder 391, the air then circulates out through the air bladder outlet 393 and is returned to the air plenum 380. Alternatively, the air could be jettisoned to atmosphere upon exiting the air bladder. In these variations, the circulating air may aid in transfer of heat (e.g., from the scalp or the chassis) away from the user. This should be more comfortable for the user, and cooling of the head (particularly the frontal cortex) has been shown to help people fall asleep more quickly. In some instances, air returned to the air plenum 380 may be heated relative to the surrounding air, which may provide warmer air to the user. This may increase the humidity and comfort of the user.

Alternatively, the air chamber could be connected by an airway passage to the flow generator. The flow generator, which provides pressurized air to the patient's airway, can also provide a flow of air to the air chamber in order to circulate the air, keeping the air chamber cool and inflated. Such a system would require an exit for the air from the chamber. This exiting air could go back to the inlet of the flow generator, or could be otherwise discharged out of the system (e.g. simply into the surrounding air).

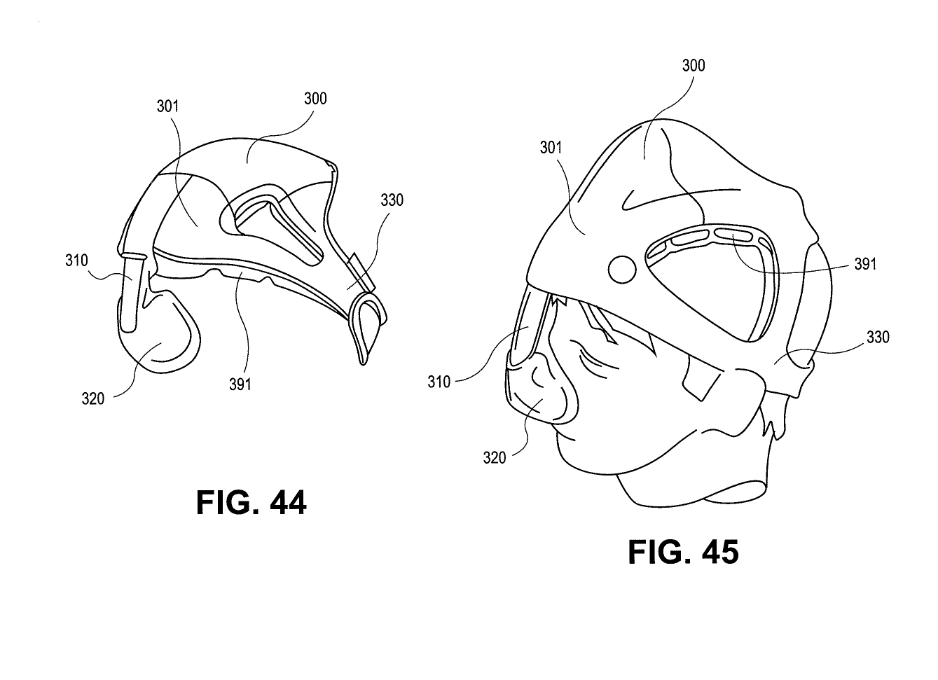

FIG. 44 is a perspective view of a wearable PAP system on a user showing many of the key elements of the features described herein. Notably, the air delivery matrix 310, the system chassis 300, the fabric cover 301, the user interface 320, and the air bladder 391 are shown together in PAP system. FIG. 45 is a perspective view of a wearable PAP system on a user showing many of the key elements of the features described herein. Notably, the air delivery matrix 310, the system chassis 300, the fabric cover 301, the user interface 320, and the air bladder 391 are shown together in PAP system.

FIG. 6 is a block diagram of the system 10 illustrating the interconnection of the various components of the system. A position signal 34 from the head position sensor 25 is transmitted to the controller 24, which in turn generates a control signal 35 for the compressor 23 based upon the received head position signal 34. The controller 24 may have an input module 36 that allows for the manual input of compressor output pressure set point(s) that provides one or more suitable compressor output pressures for the compressor for one or more received head position signals 34. The air delivery matrix 310 which delivers pressurized gas from the compressor 23 to the user interface 320 may have a pressure sensor 37 which generates a pressure signal 38 that is fed back to the controller 24 to ensure that the desired gas pressure for the sensed patient position is delivered to the patient. The pressure sensor 37 may be alternatively located in the patient's user interface 320 or the compressor discharge 29.

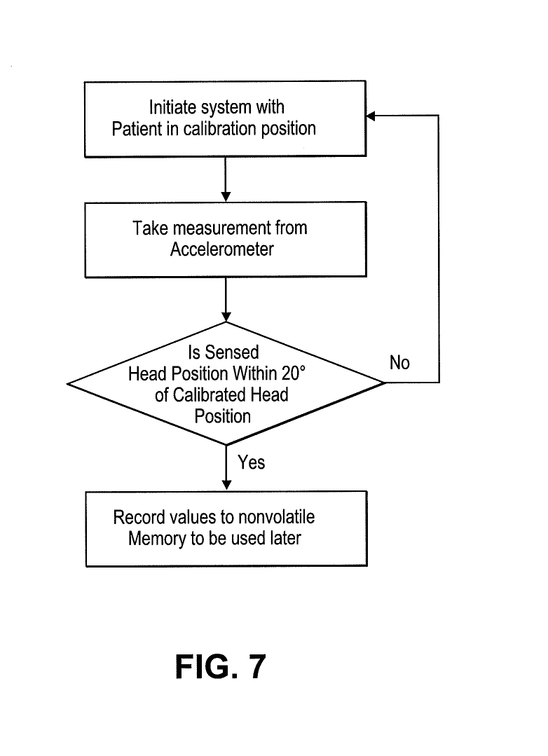

FIG. 7 is a flow diagram illustrating the calibration of the head position sensor 25. With the PAP system 10 mounted onto the patient's head, the patient reclines into a calibration position, such as the supine position, and pushes the activation button 30 on the front of the housing 11. The preferred head position for calibrating the sensor 25 is the supine position with the patient's nose is orthogonal to the sleeping plane 40. The system takes a measurement from the head position sensor 25, an accelerometer, and compares this measurement to the Gravitational constant (G). Once the measurement is within 10% of G, the value is recorded to a nonvolatile memory in the controller 24. The system 10 may be calibrated by the patient or by a health professional.

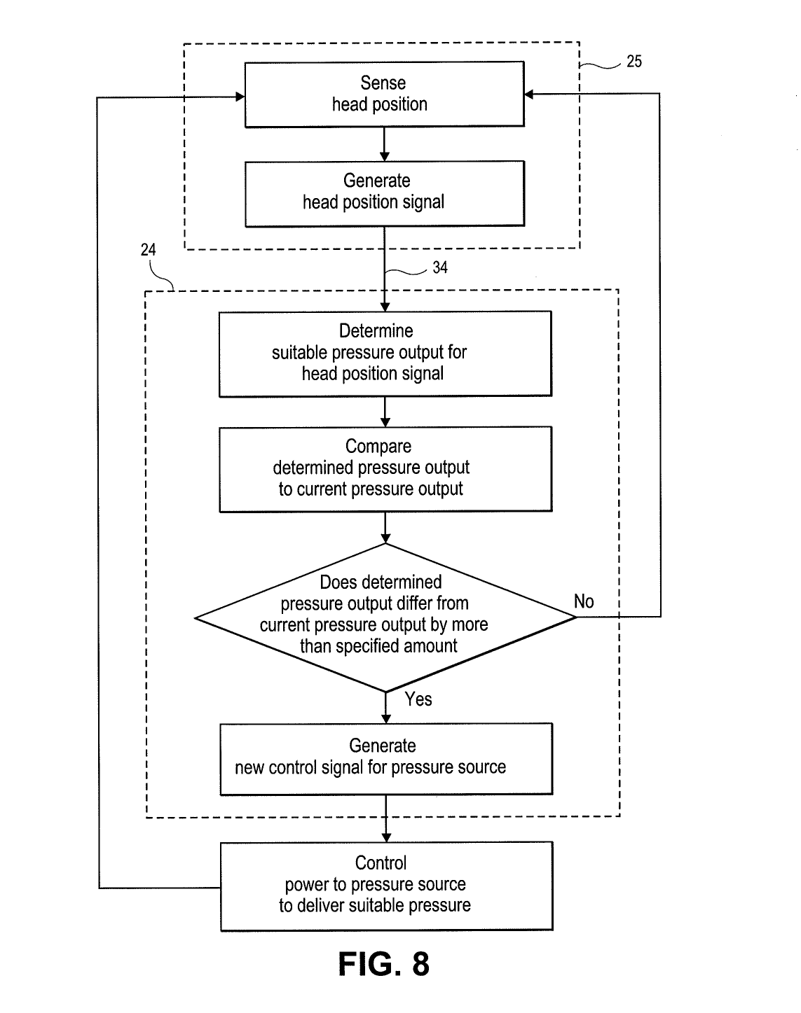

FIG. 8 is a flow diagram illustrating a variation of operation of the calibrated PAP system 10 as described here. The calibrated head position sensor 25 senses the patient's head position and generates a head position signal 34 representing then sensed head position. This signal 34 is transmitted (by a wire or wirelessly) to controller 24, compares the received head position signal 34 with a stored relationship between head position signal and compressor output pressure and determines a suitable compressor output pressure for the sensed head position. The controller 24 compares this determined pressure output to the current pressure source output pressure, and, if the determined pressure output differs from the current pressure output by more than a specified amount, then the controller will generate a new control signal for the pressure source or compressor. This new control signal will control the pressure source to deliver a suitable output pressure that is delivered to the patient or user through a user interface 320 so as to maintain a critical pressure within the patient's airway passage. If the determined pressure output does not differ from the current pressure output of the pressure source by more than a specified amount, the control signal will not be changed. The system 10 will continue to sense the head position of the patient and restart the loop.

In another embodiment, the input module 36 may be used by a health professional to input a set-point for a suitable compressor output pressure for the calibrated sensor head position that has been determined by the patient's sleep test. The controller 24 may continuously or periodically compare the sensed head position signal 34 from the position sensor 25 with the calibrated head position signal. If the comparison indicates that the patient's sensed head position deviates from the calibrated head position less than a certain amount, e.g. 20°, then the system will loop (providing the same control signal 34 to the compressor to provide a suitable compressor output pressure) until the controller detects a head position signal which represents a sensed head position that deviates more than the certain amount. When the controller 24 determines that the sensed head position deviates more that the certain amount, such as the lateral position, the controller compares the head position signal 34 with a stored relationship between head position signal and suitable compressor output pressure to determine the suitable compressor output pressure for the new sensed head position such as the lateral head position where the patient's nose lies in a plane 41 parallel to the sleeping plane 40 when the patient's head 16 is resting on pillow 42. The controller 24 then generates a new control signal 35 for the compressor to enable the compressor to provide the suitable output pressure for the sensed new lateral head position. The system 10 will loop providing the same output pressure until a new head position signal 34 indicates that the patient's head is in a new position which is more than 20° away from the lateral position.

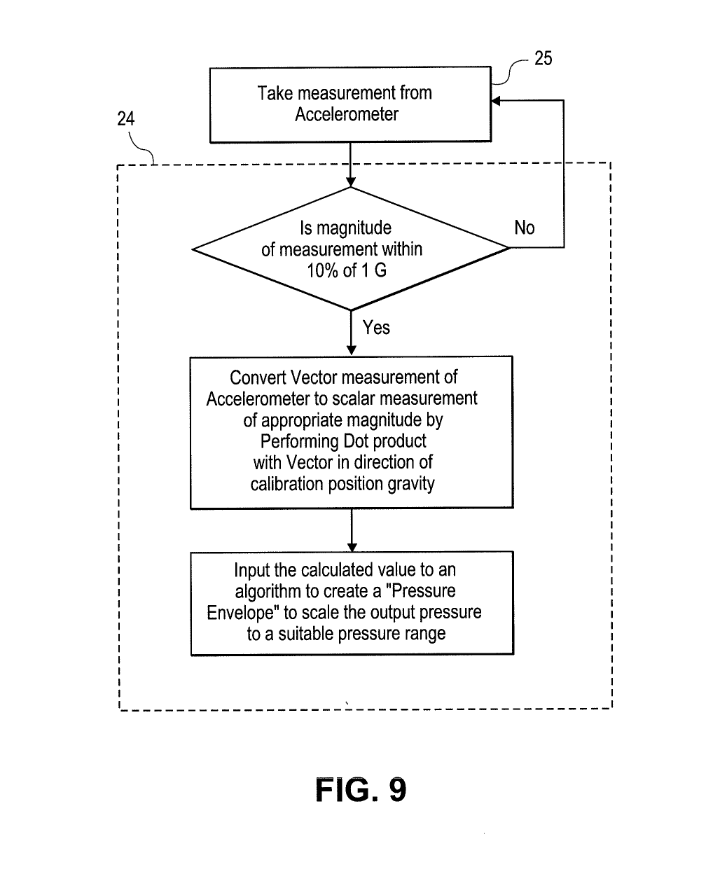

A flow chart is shown in FIG. 9 illustrating a way to scale the pressure calculation as the head is moved from the calibrated position. The position sensor 25, an accelerometer, takes a measurement which is compared with gravity to determine if the accelerometer is stabilized. If the measurement is within 10% of G, the vector measurement of the accelerometer is converted to a scalar measurement by performing a Dot product, using the vector in the direction of the calibration position gravity. Next, a minimum pressure is added to the scalar reading and then a pressure envelope is created to control the output pressure of the pressure source.

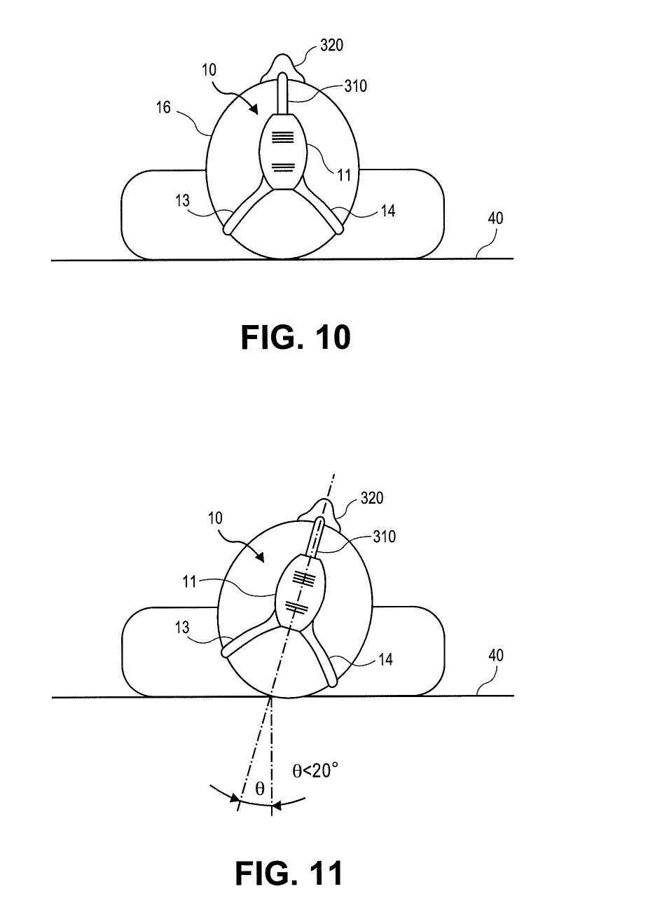

FIG. 10 shows the top view of a patient wearing a PAP system 10 as described here while lying in the supine position with the head 16 and nose pointed directly up, orthogonal to the sleeping plane 40. This is the preferred calibration position. A patient sleeping in the supine position will generally require a pressure that is higher than what is required in more lateral sleeping positions.

FIG. 11 shows a top view of a patient wearing a PAP system 10 as described here while lying with his or her torso in the supine position and the head 16 rotated slightly laterally, In this figure, angle theta represents the deviation of the head from a true supine position, here shown to be less than 20°.

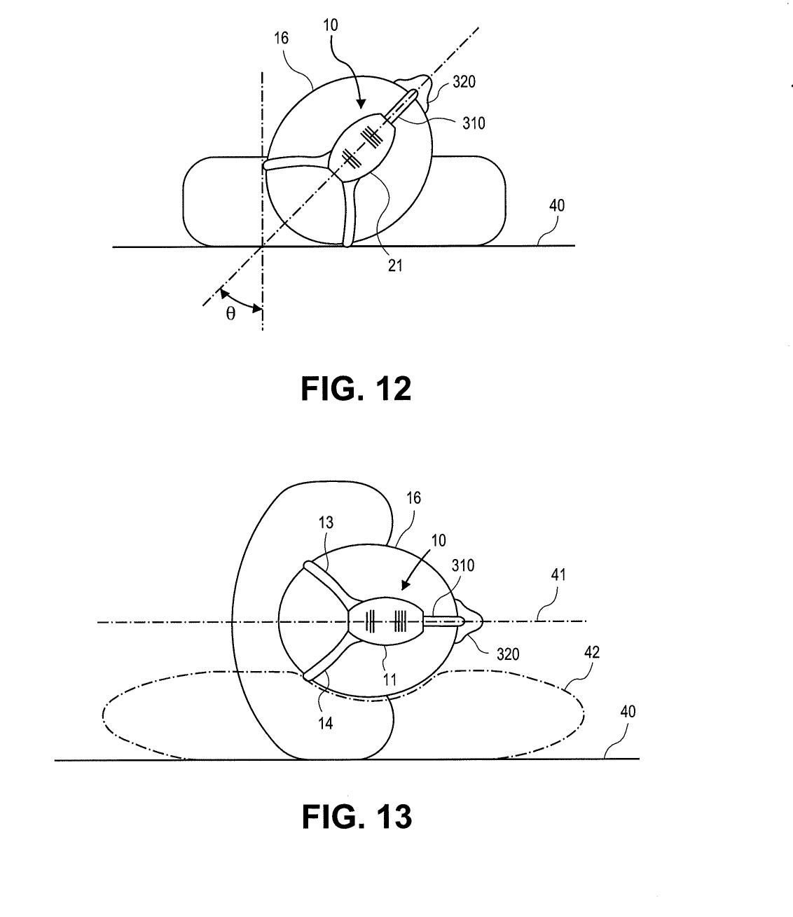

FIG. 12 shows the top view of a patient wearing a PAP system 10 as described here while lying with the patient's torso in the supine position and the patient's head 16 rotated more laterally. In this figure, angle theta represents the deviation of the head from a true supine position, here shown to be between 30° and 60°.

FIG. 13 shows a top view of a patient wearing a PAP system 10 as described here while lying with the patient's torso in the lateral position and the head 16 of the patient in a neutral lateral position on pillow 42 with the nose pointed in a plane 41 parallel to the sleeping plane 40.

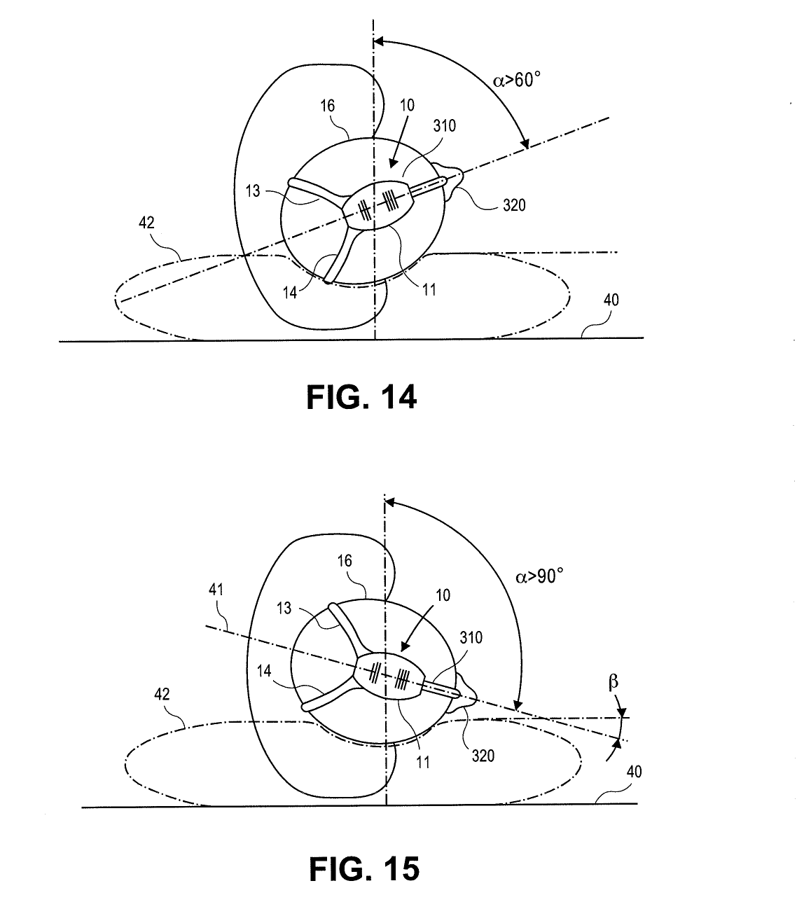

FIG. 14 shows a top view of a patient wearing a PAP system 10 as described here while lying with the torso in the lateral position and the head 16 rotated toward the supine direction relative to the lateral sleeping position which is parallel to the sleeping plane 40. Angle alpha represents the rotation of the patient's head from true supine position, here shown to be less than 90°.

FIG. 15 shows the top view of a patient wearing a PAP system 10 as described here while lying with the patient's torso in a lateral position and the patient's head 16 rotated beyond 90° from the supine position

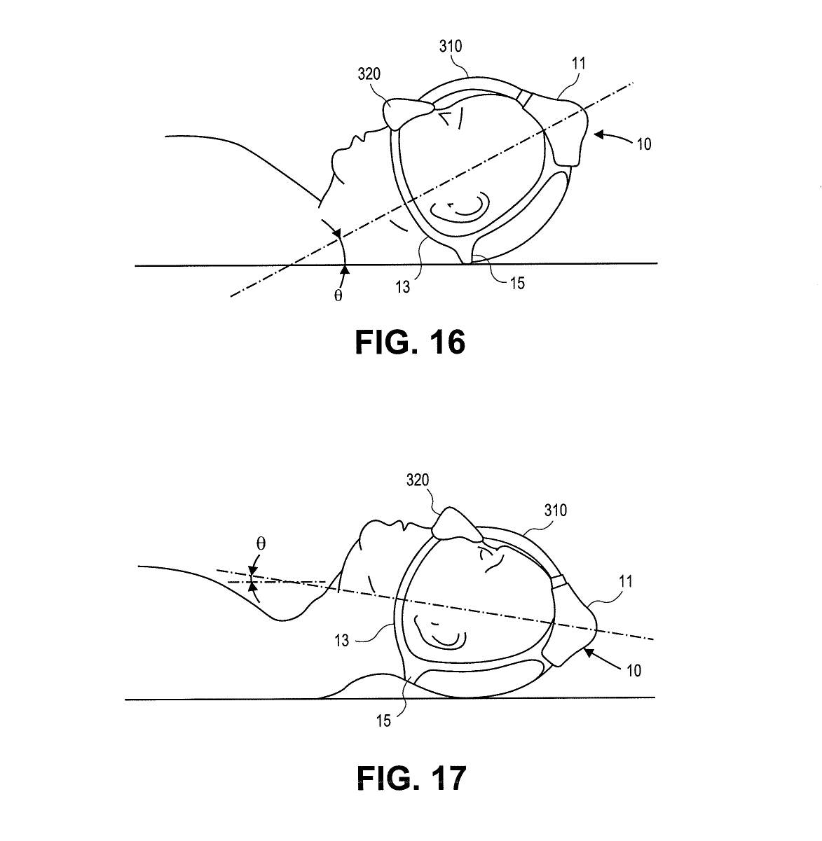

FIG. 16 shows a side view of a patient wearing a PAP system 10 as described here while lying with the patient's torso and head 16 in the supine position with the head tilted forward such that the patient's chin is closer to the patient's chest.

FIG. 17 shows a side view of a patient wearing a PAP system 10 as described here while lying with the patient's torso and head 16 in the supine position with the head tilted backward such that the patient's chin is further from the patient's chest.

The controller may be programmed to provide a suitable compressor output pressure for positions such as when the patient's head is rotated more than 90° from the supine position as shown in FIG. 15 and for positions such as when the patient's head is tilted forward or backward as shown in FIGS. 16 and 17. The stored relationship between the sensed head position signal 34 and suitable compressor output pressure may be a list of sensed head position signals with corresponding suitable compressor output pressures in a readable library in the controller 24. Alternatively, the stored relationship may be an algorithm which defines a curve of head position verses compressor output pressure.

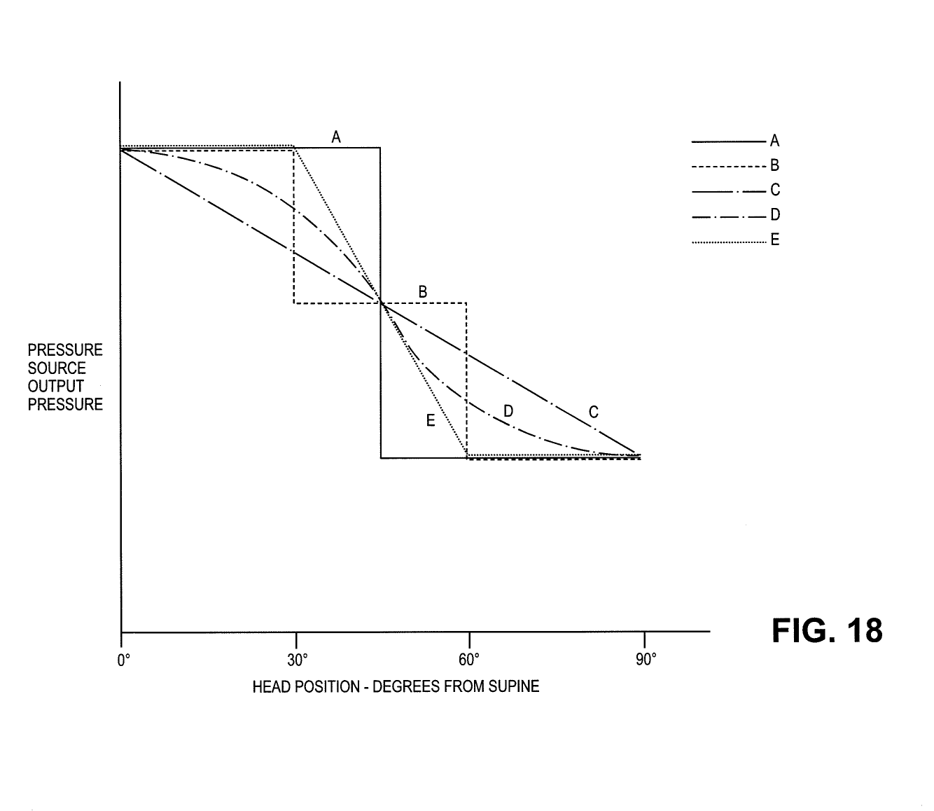

The relationship between the sensed position of the patient's head and the pressure output of the pressure source can take several forms, as depicted in the graph shown in FIG. 18. Line A depicts a step function in which, at some point between supine position and a position 90° from the supine position, the pressure output drops. Such a step function could also have multiple steps between the supine position and 90° from the supine position, as depicted in line B.

Alternatively, the relationship between head position and output pressure may follow an inclined straight line, as depicted by line C. Another possibility is shown in line D which depicts a relationship in which there is a continuous pressure drop moving from a supine position to 90° from supine, but the rate of pressure drop is greatest between 30° and 60° from the supine position. Yet another relationship is shown in line E wherein there is an inclined linear portion between the supine pressure and lateral pressure between 30° and 60°.

While not shown, the pressure could continue to drop at positions greater than 90° from the supine position. Additionally, the relationship between pressure and sensed head position may have a more elaborate, advanced curve shape to provide more appropriate pressures at each position. Other relationships may be employed.

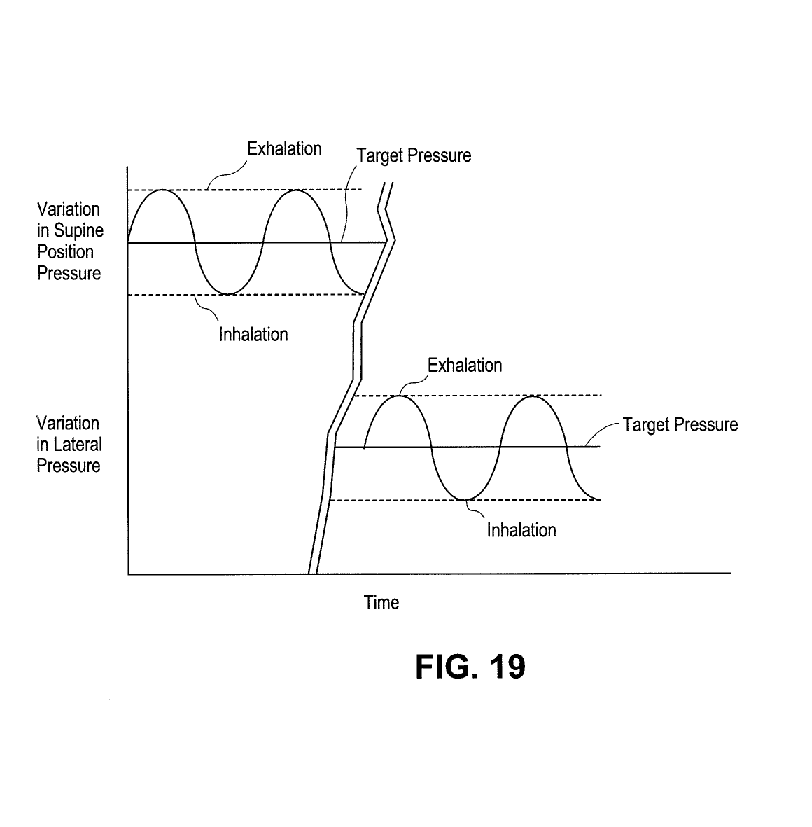

FIG. 19 graphically illustrates a respiratory cycle through two different head positions, supine and lateral. In the supine position, the delivered pressure is higher and the respiratory cycle causes the pressure to fluctuate within a range about the clinical target pressure between inhalation and exhalation. In the lateral position, the delivered pressure is lower and fluctuates between inhalation and exhalation similar to the fluctuations in the supine position.

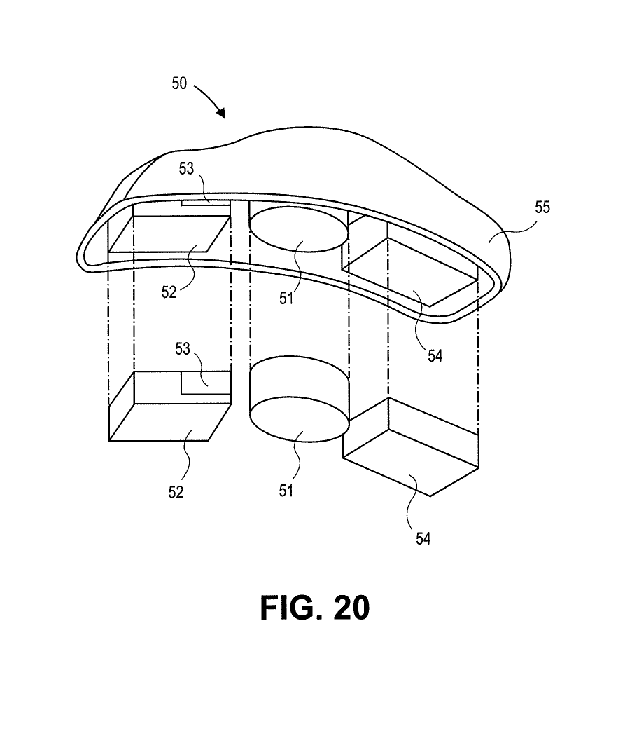

An alternative variation of a PAP system 50 as described here is shown in FIG. 20 in an exploded view. In this embodiment, the compressor 51, controller 52, position sensor 53 and battery 54 are secured to the top inner lining of the housing 55. The lower margins of housing 55 are secured to the shaded areas 56 of base 57 which may be secured to the replaceable harness assembly 58 as shown in FIG. 21. In this manner the housing 55 along with the secured components 51-54 could be reusable, whereas the harness assembly 57 and 58 which has direct contact with the patient can be easily replaced as needed. The shaded contact areas 56 of the base 57 may be recessed so as to provide a better fit for the lower margins of the housing 55.

Another alternative embodiment is shown in FIG. 22 wherein a PAP system 60 as described here has multiple housings, housing 61 which holds the compressor and housing 62 which holds the controller and battery. Housings 61 and 62 are secured to the harness assembly 63 which holds the system 60 against the patient's head. The position sensor is secured to the interior of one of the housings, preferably to the controller that is secured to the housing 62. With this particular configuration of the PAP system 60, the weight of the PAP system can be more evenly distributed over the top of the patient's head. Moreover, a smaller area of the patient's head is covered with the housing which improves the comfort and heat regulation and reduces potential irritation of the patient's scalp. Additionally, the harness assembly 63 may have a flexible base 64 and the multiple housings 61 and 62 secured to the flexible base can better conform to the shape of the patient's head and facilitate easier replacement of the various components of the system. Other advantages are apparent.

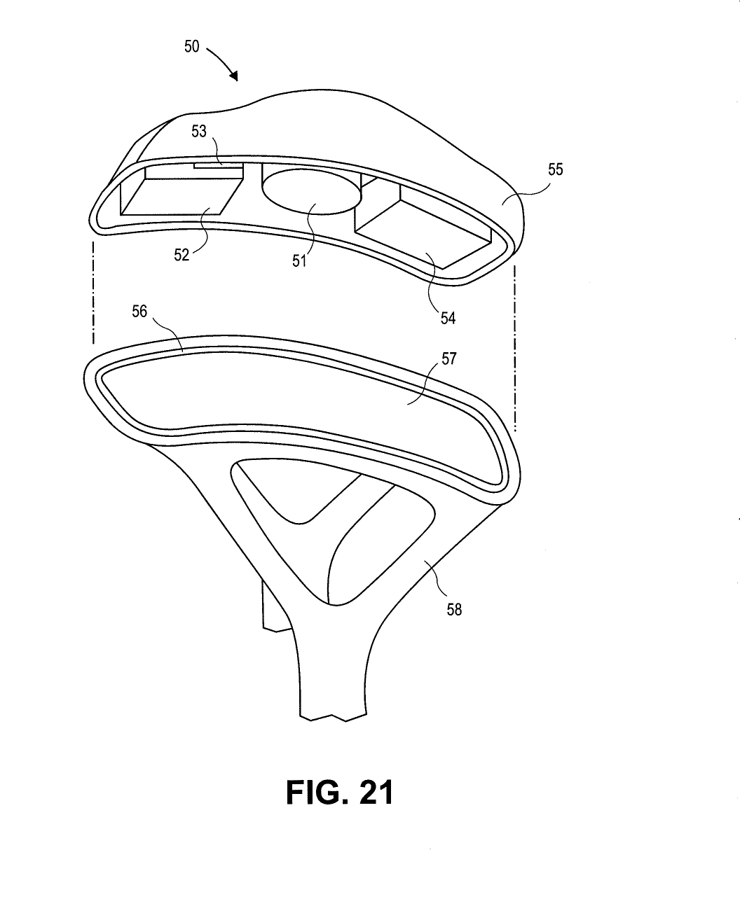

FIG. 23 illustrates yet another alternative PAP system 70 having multiple housings which contain system components. In PAP system 70, housing 71 contains the pressure source and motor drive (not shown) and housing 72 contains the controller (not shown). The electrical power source is a plurality of battery cells 73 which are secured to the harness assembly 74, preferably to a flexible base 75 which is part of the harness assembly. The harness assembly 74 secures the multiple housings 71 and 72 and battery cells 73 to the patient's head. Conductor wire(s) (not shown) interconnect the battery cells 73 and the motor drive for the compressor and the controller and the motor drive for the compressor.

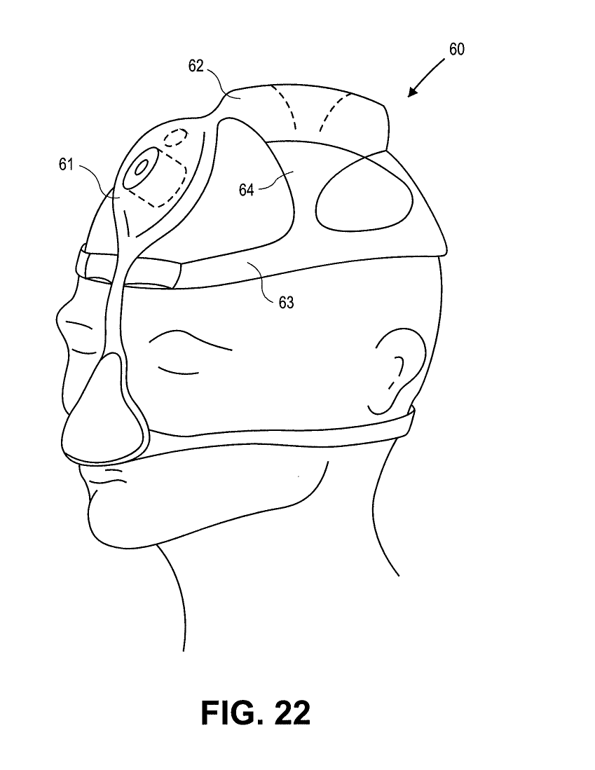

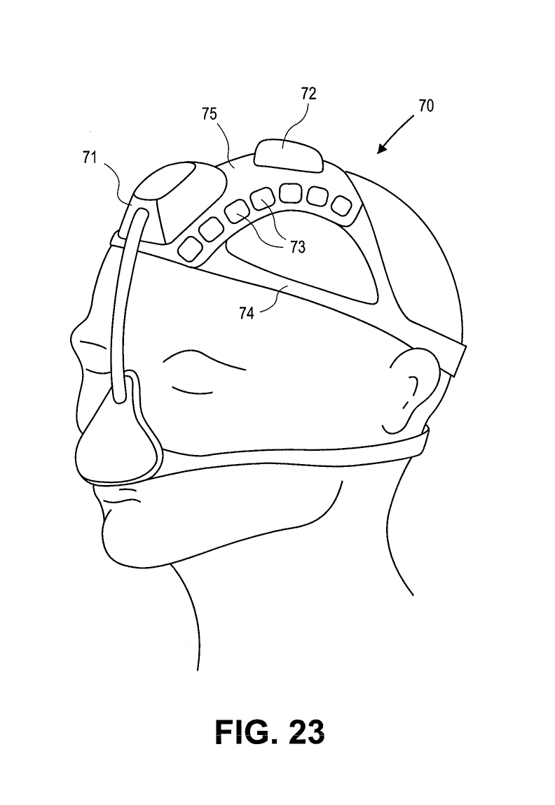

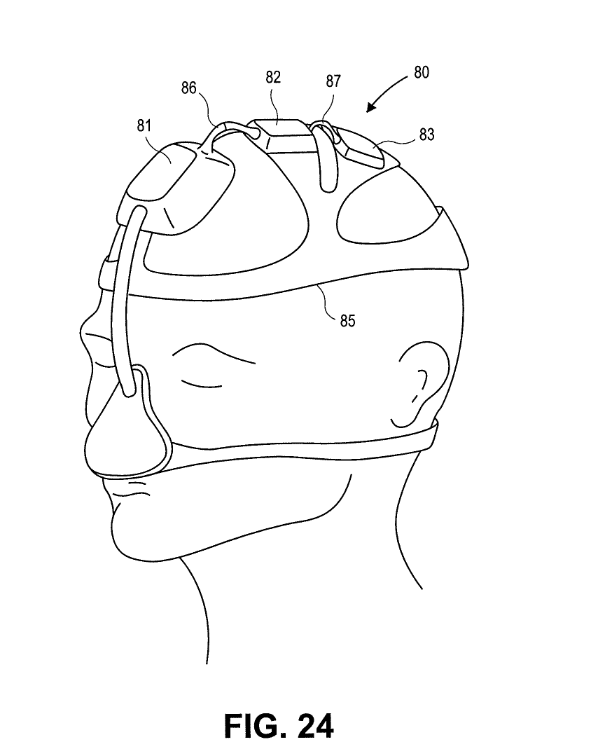

FIG. 24 illustrates yet another alternative PAP system 80 having multiple housings which contain system components. In PAP system 80, housing 81 contains the pressure source and motor drive (not shown) and housing 82 contains the controller (not shown). Housing 83 contains the electrical power source such as one or more batteries (not shown). The plurality of housings 81-83 are secured to a harness assembly 85. The individual housings 81-83 are interconnected by flexible connections or joints 86 and 87. The harness assembly 85 secures the multiple housings 81, 82 and 83 to the patient's head. Conductor wire(s) (not shown) interconnect the electrical power source and the motor drive for the compressor and conductor wires (not shown) connect the controller and the motor drive for the pressure source.

While particular forms of the invention have been illustrated and described herein, it will be apparent that various modifications and improvements can be made to the invention. For example, while the description herein has focused on PAP systems, the system may be utilized in a variety of breathing systems. Additionally, the PAP systems are primarily described herein as self-contained breathing systems. However, many of the advantageous features described herein may be applicable to breathing systems with remote control and/or pressure sources and wherein the head position sensor is secured to the top of the patient's head. To the extent not otherwise described herein, materials and structure may be of conventional design.

Moreover, individual features of embodiments of the devices and methods may be shown in some drawings and not in others, but those skilled in the art will recognize that individual features of one embodiment of the devices and methods can be combined with any or all the features of another embodiment. Accordingly, it is not intended that the devices and methods be limited to the specific embodiments illustrated.

1. A positive airway pressure system comprising:
a wearable user interface for delivery of pressurized air to a user;
a pressure source configured to provide the pressurized air; and
an air delivery matrix between the user interface and the pressure source, wherein the air delivery matrix is configured to transmit the pressurized air from the pressure source to the user interface through a non-hollow portion of the air delivery matrix.

2. The system of claim 1 wherein the wearable user interface comprises a mask.

3. The system of claim 1 wherein the air delivery matrix comprises an inner matrix enclosed in an outer matrix, and wherein the air delivery matrix is configured to transmit the pressurized air through the inner matrix.

4. The system of claim 3 wherein the outer matrix comprises a first porous material and the inner matrix comprises a second porous material, and wherein the second porous material has lower resistance to airflow than the first porous material.

5. (canceled)

6. The system of claim 4 wherein a density of the second porous material may vary along a length of the inner matrix.

7. The system of claim 3 wherein the air delivery matrix further comprises a separation layer between the inner matrix and the outer matrix.

8. The system of claim 4 wherein the second porous material is configured to allow one or more gases to pass through the outer matrix, from the inner matrix out of the air delivery matrix.

9. The system of claim 3 wherein the outer matrix comprises one or more support members extending along a length of the outer matrix.

10. The system of claim 3 wherein the outer matrix comprises one or more lumens extending therethrough, and wherein the system is configured to measure a pressure in the user interface using the one or more lumens.

11. The system of claim 1 wherein the air delivery matrix is tapered between a narrow end and wide end.

12. (canceled)

13. (canceled)

14. The system of claim 3 wherein the inner matrix comprises a first inner matrix and a second inner matrix, wherein the outer matrix separates the first inner matrix and the second inner matrix.

15. The system of claim 1 wherein the pressure source is at least partially housed within a chassis configured to be coupled to a head of the user.

16. The system of claim 15 wherein at least a portion of the chassis is formed from a foam material.

17. The system of claim 15 wherein the chassis comprises one or more air inlets and one or more air filters covering the one or more air inlets.

18. (canceled)

19. (canceled)

20. (canceled)

21. (canceled)

22. (canceled)

23. (canceled)

24. A method for providing pressurized air to a patient's airway comprising:
delivering pressurized air to a patient using an air pressure system comprising:
a wearable user interface for delivery of pressurized air to a user;
a pressure source configured to provide the pressurized air; and
an air delivery matrix between the user interface and the pressure source, wherein the air delivery matrix is configured to transmit the pressurized air from the pressure source to the user interface through a non-hollow portion of the air delivery matrix.

25. (canceled)

26. The method of claim 24 wherein the air delivery matrix comprises an inner matrix enclosed in an outer matrix, and wherein the air delivery matrix is configured to transmit the pressurized air through the inner matrix.

27. The method of claim 26 wherein the outer matrix comprises a first porous material and the inner matrix comprises a second porous material, and wherein the second porous material has lower resistance to airflow than the first porous material.

28. (canceled)

29. (canceled)

30. (canceled)

31. The method of claim 27 wherein the second porous material is configured to allow one or more gases to pass through the outer matrix, from the inner matrix out of the air delivery matrix.

32. (canceled)

33. The method of claim 26 wherein the outer matrix comprises one or more lumens extending therethrough, and wherein the system is configured to measure a pressure in the user interface using the one or more lumens.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. The system of claim 3 wherein the inner matrix comprises one or more support members extending along a length of the inner matrix.

48. (canceled)
